(19) United States
(12) Patent Application Publication
(10) Pub. No.: US 2026/0191507 A1
(43) Pub. Date: Jul. 9, 2026

(54) ULTRASOUND IMAGING APPARATUS AND METHOD OF VISUALIZING ULTRASOUND MERGED VOLUME

(71) Applicant: Samsung Medison Co., Ltd., Hongcheon-gun (KR)

(72) Inventors: Hyeoncheol Jo, Seoul (KR); Hanjun Kim, Seoul (KR); Byungyeon Kim, Seoul (KR); Gyuha Park, Seoul (KR); Teajun Jang, Seoul (KR)

(73) Assignee: SAMSUNG MEDISON CO., LTD., Hongcheon-gun (KR)

(21) Appl. No.: 19/323,553

(22) Filed: Sep. 9, 2025

(30) Foreign Application Priority Data
Jan. 7, 2025 (KR) 10-2025-0002398

(51) Int. Cl. A61B 8/00; A61B 8/08; G06T 15/08

(52) U.S. Cl. CPC A61B 8/463; A61B 8/0808; A61B 8/0866; G06T 15/08; G06T 2210/41

(57) ABSTRACT

Provided are an ultrasound imaging apparatus and a method of visualizing an ultrasound merged volume. The ultrasound imaging apparatus includes an ultrasound transceiving module, a memory storing instructions, and at least one processor comprising processing circuitry, wherein, when the instructions are individually or collectively executed by the at least one processor, the ultrasound imaging apparatus is configured to obtain a plurality of original volumes through the ultrasound transceiving module, generate a merged volume by merging the plurality of original volumes, display a merged volume cross-section image indicating a cross-sectional area of interest within the merged volume, receive a user's input to select one of the plurality of original volumes, and based on a position of the cross-sectional area of interest within the merged volume, display an original volume cross-section image of the selected original volume corresponding to the merged volume cross-section image.

### CROSS-REFERENCE TO RELATED APPLICATION

This application is based on and claims priority under 35 U.S.C. § 119 to Korean Patent Application No. 10-2025-0002398, filed on Jan. 7, 2025, in the Korean Intellectual Property Office, the disclosure of which is incorporated by reference herein in its entirety.

### BACKGROUND

### 1. Field

The disclosure relates to an ultrasound imaging apparatus for visualizing an ultrasound merged volume, a method, performed by the ultrasound imaging apparatus, of visualizing an ultrasound merged volume, and a computer-readable recording medium storing a computer program for performing the method.

### 2. Description of the Related Art

Recently, in the medical field, various medical imaging apparatuses have been widely used to obtain information about biological tissues of the human body through imaging, for the purpose of early diagnosis or surgical procedures of various diseases. Typical examples of medical imaging apparatuses include ultrasound imaging apparatuses, computed tomography (CT) apparatuses, and magnetic resonance imaging (MRI) apparatuses.

An ultrasound imaging apparatus is a device that noninvasively obtains at least one image of an internal region of an object (e.g., soft tissue or blood flow) by irradiating an object with an ultrasound signal generated from a transducer of a probe and receiving information from the signal reflected by the object. The ultrasound imaging apparatus may be used for medical purposes such as observing the internal structure of an object, detecting foreign materials, and measuring injuries. This ultrasound imaging apparatus has advantages of high stability, real-time image display capability, and safety due to the absence of radiation exposure, compared to an imaging apparatus using X-rays. Accordingly, the ultrasound imaging apparatus is widely used in conjunction with other imaging apparatuses.

### SUMMARY

Additional aspects will be set forth in part in the description which follows and, in part, will be apparent from the description, or may be learned by practice of the presented embodiments of the disclosure.

According to an aspect of the disclosure, provided is an ultrasound imaging apparatus. The ultrasound imaging apparatus includes an ultrasound transceiving module, a memory storing instructions, and at least one processor including processing circuitry. When the instructions are individually or collectively executed by the at least one processor, the ultrasound imaging apparatus is configured to obtain a plurality of original volumes through the ultrasound transceiving module, generate a merged volume by merging the plurality of original volumes, display a merged volume cross-section image indicating a cross-sectional area of interest within the merged volume, receive a user's input to select one of the plurality of original volumes, and based on a position of the cross-sectional area of interest within the merged volume, display an original volume cross-section image of the selected original volume corresponding to the merged volume cross-section image.

According to another aspect of the disclosure, provided is a method of visualizing an ultrasound merged volume. The method of visualizing an ultrasound merged volume includes obtaining a plurality of original volumes, generating a merged volume by merging the plurality of original volumes, displaying a merged volume cross-section image indicating a cross-sectional area of interest within the merged volume, receiving a user's input to select one of the plurality of original volumes, and displaying an original volume cross-section image of the selected original volume corresponding to the merged volume cross-section image based on a position of the cross-sectional area of interest within the merged volume.

According to another aspect of the disclosure, provided is a computer-readable recording medium having recorded thereon a program for performing, on a computer, the method of visualizing an ultrasound merged volume.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A and 1B are block diagrams showing the configuration of an ultrasound imaging system according to an embodiment;

FIGS. 2A, 2B, 2C, and 2D are diagrams illustrating an ultrasound imaging system according to an embodiment;

FIG. 3 is a diagram showing a method, performed by an ultrasound imaging apparatus, of displaying data from a merged volume, according to an embodiment;

FIG. 4 is a flowchart of a method, performed by an ultrasound imaging apparatus, of displaying data from a merged volume, according to an embodiment;

FIG. 5 is a diagram showing a method, performed by an ultrasound imaging apparatus, of registering or stitching original volumes, according to an embodiment;

FIG. 6 is a diagram showing a method, performed by an ultrasound imaging apparatus, of displaying cross-sections A, B, and C of an ultrasound volume, according to an embodiment;

FIG. 7 is a diagram showing a method, performed by an ultrasound imaging apparatus, of displaying a cross-sectional image of a registered volume, according to an embodiment;

FIG. 8 is a diagram showing a method, performed by an ultrasound imaging apparatus, of displaying a cross-sectional image of a merged volume, according to an embodiment;

FIGS. 9A and 9B are diagrams showing a method, performed by an ultrasound imaging apparatus, of providing a user interface for selecting a cross-sectional image of an original volume, according to an embodiment;

FIG. 10 is a flowchart showing a method, performed by an ultrasound imaging apparatus, of displaying a cross-sectional image of a merged volume based on priorities of original volumes, according to an embodiment;

FIG. 11 is a diagram showing a method, performed by an ultrasound imaging apparatus, of displaying cross-sections A, B, and C of a stitched volume, according to an embodiment;

FIG. 12 is a diagram showing a method, performed by an ultrasound imaging apparatus, of displaying a cross-sectional image of a stitched volume based on priorities of original volumes, according to an embodiment;

FIG. 13 is a diagram showing a method, performed by an ultrasound imaging apparatus, of selecting the priorities of original volumes for one cross-sectional image, according to an embodiment;

FIGS. 14A and 14B are diagrams showing a method, performed by an ultrasound imaging apparatus, of displaying a cross-sectional image of a merged volume, according to an embodiment;

FIG. 15 is a diagram showing a method, performed by an ultrasound imaging apparatus, of displaying a cross-sectional image of an original volume corresponding to the cross-section of a merged volume, according to an embodiment; and

FIG. 16 is a diagram showing a method, performed by an ultrasound imaging apparatus, of providing a cross-sectional image of a merged volume based on priorities of original volumes, according to an embodiment.

### DETAILED DESCRIPTION

In the disclosure, the expression “at least one of a, b, or c” may refer to “a,” “b,” “c,” “a and b,” “a and c,” “b and c,” “all of a, b, and c,”, or modifications thereof.

Hereinafter, embodiments of the disclosure will be described in detail with reference to the accompanying drawings so that those skilled in the art may easily carry out the disclosure. However, the disclosure may be implemented in various different forms and is not limited to the embodiments described herein. In addition, in the drawings, parts not directly related to the description have been omitted for clarity of the disclosure, and like reference numerals are used for like elements throughout the specification.

The terms used in the disclosure are selected in consideration of functions described in the disclosure and are generally used terms. However, these terms may vary depending on the intention of those skilled in the art, court precedents, or the emergence of new technologies. Accordingly, the terms used herein should not be interpreted based solely on their literal meanings but should be construed based on the meanings intended in the disclosure and the overall content of the specification.

In addition, terms such as “first” and “second” may be used to describe various components, but such terms shall not be construed as limiting the components. These terms are merely used to distinguish one component from another.

Furthermore, the terms used in the disclosure are used solely for the purpose of describing particular embodiments and are not intended to limit the scope of the disclosure. Unless clearly indicated otherwise from the context, the singular forms used herein include plural references. In the specification, when a certain part is described as being “connected” to another part, it is to be understood that the connection may be either a “direct connection” or an “indirect electrical connection” via an intermediate component. Also, when a part is described as “including” a certain component, it is to be understood, unless explicitly stated otherwise, that the part may further include other components in addition to the specified component.

It is to be understood that not all elements of the embodiments are described in the present specification, and general content or redundant content between embodiments that would be obvious to one of ordinary skill in the art is omitted. The terms “module” or “unit” used in the present specification may be implemented as software, hardware, or firmware, or a combination of two or more thereof. According to embodiments, a plurality of “modules” or “units” may be implemented as a single element, or a single “module” or “unit” may include a plurality of elements.

Expressions such as “in some embodiments” or “in an embodiment” appearing throughout the specification do not necessarily refer to the same embodiment. The disclosure can be readily understood by combining the following detailed description with accompanying drawings, and reference numerals refer to structural elements.

In the disclosure, the term “object” refers to a subject to be imaged and may include a human, an animal, or a part thereof. For example, the object may include a part of a body (e.g., an organ or a body structure) or a phantom.

In the disclosure, the term “ultrasound image” refers to an image of the object that is generated or processed based on ultrasound signals transmitted to and reflected from the object.

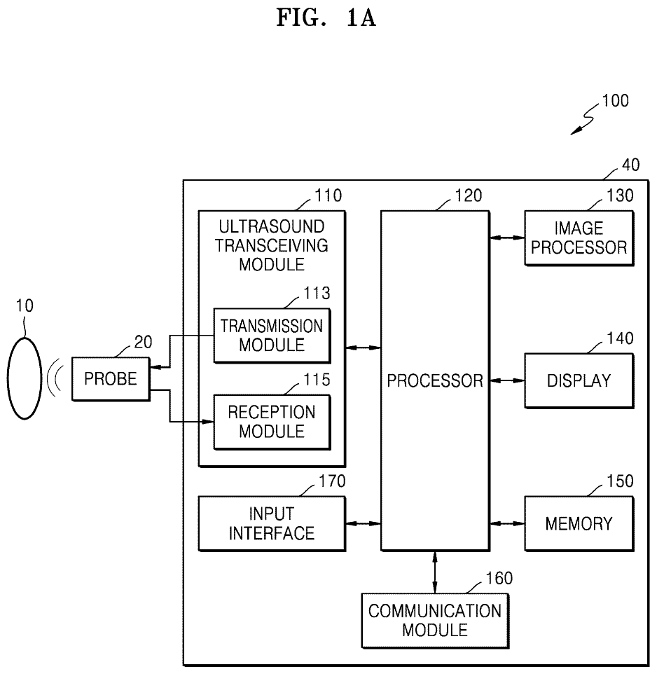

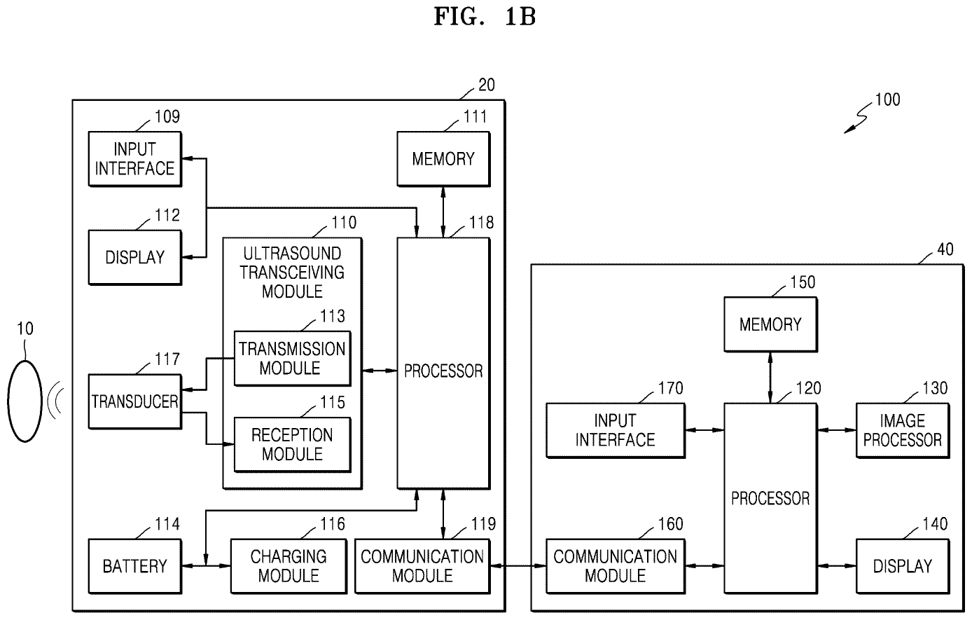

Referring to FIGS. 1A and 1B, an ultrasound imaging system 100 may include a probe 20 and an ultrasound imaging apparatus 40.

The ultrasound imaging apparatus 40 may be implemented not only as a cart type but also as a portable type. Examples of a portable ultrasound imaging apparatus may include a probe, a smart phone including an application, a laptop computer, a personal digital assistant (PDA), or a tablet personal computer (PC), but the disclosure is not limited thereto. The ultrasound imaging apparatus 40 may be implemented as a probe integrated type.

The probe 20 may include a wired probe that is connected to the ultrasound imaging apparatus 40 via a wired connection and communicates with the ultrasound imaging apparatus 40 through the wired connection, a wireless probe that is connected to the ultrasound imaging apparatus 40 via a wireless connection and communicates with the ultrasound imaging apparatus 40 through the wireless connection, and/or a hybrid probe that is connected to the ultrasound imaging apparatus 40 via a wired or wireless connection and communicates with the ultrasound imaging apparatus 40 through the corresponding connection.

According to various embodiments, as illustrated in FIG. 1A, the ultrasound imaging apparatus 40 may include an ultrasound transceiving module 110, and as illustrated in FIG. 1B, the probe 20 may include the ultrasound transceiving module 110. According to various embodiments, both of the ultrasound imaging apparatus 40 and the probe 20 may include the ultrasound transceiving module 110.

According to various embodiments, the probe 20 may further include at least one of an image processor 130, a display 140, an input interface 170, or a combination thereof. In the disclosure, the description on the ultrasound transceiving module 110, the image processor 130, the display 140, or the input interface 170 included in the ultrasound imaging apparatus 40 may be applied to the ultrasound transceiving module 110, the image processor 130, the display 140, or the input interface 170 included in the probe 20.

FIG. 1A is a block diagram showing a configuration of the ultrasound imaging system 100 when the probe 20 is a wired probe or a hybrid probe.

The probe 20 may include a plurality of transducers. The plurality of transducers may be arranged in a certain array and implemented as a transducer array. The transducer array may correspond to a one-dimensional (1D) array or a two-dimensional (2D) array. The plurality of transducers may transmit ultrasound signals to an object 10 based on transmission signals applied by a transmission module 113. The plurality of transducers may receive ultrasound signals (echo signals) reflected from the object 10 and generate reception signals. Furthermore, the probe 20 may be implemented as an integrated type with the ultrasound imaging apparatus 40 or as a separated type connected to the ultrasound imaging apparatus 40 by a wire. Furthermore, the ultrasound imaging apparatus 40 may be connected to one or a plurality of the probe 20 based on the implementation type.

When the probe 20 is a wired probe or a hybrid probe, the probe 20 may include a connector or a cable that is connectable to a connector of the ultrasound imaging apparatus 40.

The probe 20 according to an embodiment may be implemented as a two-dimensional probe. When the probe 20 is implemented as a two-dimensional probe, the plurality of transducers included in the probe 20 may be arranged two-dimensionally to form a two-dimensional transducer array.

For example, the two-dimensional transducer array may be in a form in which a plurality of sub-arrays, each including a plurality of transducers arranged in a first direction, are arranged in plurality in a second direction different from the first direction.

Furthermore, according to an embodiment, when the probe 20 is implemented as a two-dimensional probe, the ultrasound transceiving module 110 may include at least one of an analog beamformer or a digital beamformer. Furthermore, according to an embodiment, the two-dimensional probe may include at least one of an analog beamformer, a digital beamformer, or a combination thereof, based on the implementation type.

Considering the positions and focal points of the plurality of transducers included in the probe 20, a processor 120 may control the transmission module 113 to generate transmission signals to be applied to the respective transducers.

The processor 120 may control a reception module 115 to generate ultrasound data by performing analog-to-digital conversion on a reception signal received from the probe 20, and summing the digitally converted reception signal considering the positions and focal points of the plurality of transducers.

When the probe 20 is implemented as a two-dimensional probe, the processor 120 may calculate a time delay value for digital beamforming for each sub-array, with respect to each of the plurality of sub-arrays included in the two-dimensional transducer array. Furthermore, the processor 120 may calculate a time delay value for analog beamforming with respect to each of transducers included in any one of the plurality of sub-arrays. The processor 120 may control an analog beamformer and a digital beamformer to generate transmission signals to be applied to the respective transducers based on the time delay value for analog beamforming and the time delay value for digital beamforming. Furthermore, the processor 120 may control the analog beamformer to sum the signals received from the plurality of transducers, for each sub-array, based on the time delay value for analog beamforming. Furthermore, the processor 120 may control the ultrasound transceiving module 110 to perform analog-to-digital conversion on the signal summed for each sub-array. Furthermore, the processor 120 may control the digital beamformer to generate ultrasound data by summing the digitally converted signals based on the time delay value for digital beamforming.

The image processor 130 may generate or process an ultrasound image by using the generated ultrasound data.

The display 140 may display the generated ultrasound image and various pieces of information processed in the ultrasound imaging apparatus 40 or the probe 20. The probe 20 or the ultrasound imaging apparatus 40 may include one or a plurality of displays 140 based on the implementation type. Furthermore, the display 140 may include a touch panel or a touch screen. Furthermore, the display 140 may include a flexible display.

The processor 120 may control the overall operation of the ultrasound imaging apparatus 40, and may control the operation of components of the ultrasound imaging apparatus 40. The processor 120 may perform or control various operations or functions of the ultrasound imaging apparatus 40, by performing a program or instructions stored in a memory 150. Furthermore, the processor 120 may control the operation of the ultrasound imaging apparatus 40 by receiving a control signal from the input interface 170 or an external device.

The ultrasound imaging apparatus 40 may include a communication module 160, and may be connected to an external device (e.g., the probe 20, a server, a medical device, or a portable device, such as a smartphone, a tablet PC, or a wearable device) to communicate therewith through the communication module 160.

The communication module 160 may include one or more components to enable communication with the external device. The communication module 160 may include at least one of, for example, a short-range communication module, a wired communication module, or a wireless communication module.

The communication module 160 may receive a control signal or data from the external device. The processor 120 may control the operation of the ultrasound imaging apparatus 40 based on the control signal received through the communication module 160. Furthermore, the processor 120 may transmit a control signal to the external device through the communication module 160 to control the external device based on the transmitted control signal. The external device may operate based on the control signal received from the ultrasound imaging apparatus 40 or process data received from the ultrasound imaging apparatus 40.

A program or application related to the ultrasound imaging apparatus 40 may be installed in the external device. The program or application installed in the external device may control the ultrasound imaging apparatus 40 or may be executed based on the control signal or data received from the ultrasound imaging apparatus 40.

The external device may receive or download programs or applications related to the ultrasound imaging apparatus 40 from the ultrasound imaging apparatus 40, the probe 20, or the server, and install the programs or applications in the external device to execute the installed programs or applications. The ultrasound imaging apparatus 40, the probe 20, or the server that provides the program or application may include a recording medium for storing instructions, commands, installation files, execution files, or relevant data of the program or application. The external device may be sold with the program or application installed.

The memory 150 may store various pieces of data or programs to drive and control the ultrasound imaging apparatus 40, input/output ultrasound data, or ultrasound images.

The input interface 170 may receive a user's input to control the ultrasound imaging apparatus 40. For example, the user's input may include an input for manipulating a button, a key pad, a mouse, a trackball, a jog switch, or a knob, an input for touching a touch pad or a touch screen, a voice input, a motion input, or a biometric information input (e.g., iris recognition or fingerprint recognition), but the disclosure is not limited thereto.

The at least one processor 120 may include processing circuitry. As the instructions in the memory 150 are individually or collectively executed by the at least one processor 120, the ultrasound imaging apparatus 40 may perform an embodiment.

The at least one processor 120 may obtain a plurality of original volumes through the ultrasound transceiving module 110. The at least one processor 120 may generate a merged volume by merging the plurality of original volumes.

The at least one processor 120 may display, through the display 140, a merged volume cross-section image indicating a cross-sectional area of interest within the merged volume.

The at least one processor 120 may receive, through the input interface 170, a user's input to select one of the plurality of original volumes.

The at least one processor 120 may display an original volume cross-section image of a selected original volume corresponding to the merged volume cross-section image, based on the position of the cross-sectional area of interest within the merged volume.

The at least one processor 120 may obtain an original volume cross-section image of a selected original volume corresponding to the merged volume cross-section image, based on the position of the cross-sectional area of interest within the merged volume. The at least one processor 120 may display, through the display 140, the obtained original volume cross-section image.

The at least one processor 120 may update the merged volume cross-section image so as to preferentially display data of the selected original volume for the area where the data of the original volume selected from the cross-sectional area of interest within the merged volume overlaps data of the other original volumes.

The at least one processor 120 may receive, through the input interface 170, a user's input to set the priority of the plurality of original volumes.

The at least one processor 120 may update the merged volume cross-section image so as to preferentially display data of an original volume with relatively high priority for the area where the data of the plurality of original volumes overlap.

The at least one processor 120 may receive, through the input interface 170, a user's input to set a reference volume from among the plurality of original volumes. The at least one processor 120 may merge the plurality of original volumes based on the coordinate system of the reference volume.

The at least one processor 120 may receive, through the input interface 170, a user's input to set the cross-sectional area of interest within the merged volume.

The at least one processor 120 may display, through the display 140, a plurality of merged volume cross-section images indicating a plurality of cross-sectional areas of interest within the merged volume.

The at least one processor 120 may display, through the display 140, a plurality of original volume cross-section images of the selected original volume corresponding to the plurality of merged volume cross-section images.

The at least one processor 120 may display, through the display 140, a user interface for selecting one of the plurality of original volumes.

FIG. 1B is a control block diagram of the ultrasound imaging system 100 when the probe 20 is a wireless probe or a hybrid probe.

According to various embodiments, the ultrasound imaging apparatus 40 illustrated in FIG. 1B may be substituted with the ultrasound imaging apparatus 40 described with reference to FIG. 1A.

According to various embodiments, the probe 20 illustrated in FIG. 1A may be substituted with the probe 20 to be described with reference to FIG. 1B.

The probe 20 may include a display 112, the transmission module 113, a battery 114, a transducer 117, a charging module 116, the reception module 115, an input interface 109, a processor 118, and a communication module 119. Although FIG. 1B illustrates that the probe 20 includes both of the transmission module 113 and the reception module 115, based on the implementation type, the probe 20 may include only one of the transmission module 113 and the reception module 115, and the other of the transmission module 113 and the reception module 115 may be included in the ultrasound imaging apparatus 40. Furthermore, according to an embodiment, the probe 20 may further include the image processor 130.

The transducer 117 may include a plurality of transducers. The plurality of transducers may be arranged in a certain array and implemented as a transducer array. The transducer array may corresponding to a 1D array or a 2D array. The plurality of transducers transmit ultrasound signals to the object 10 based on the transmission signal applied by the transmission module 113. Furthermore, the plurality of transducers may receive ultrasound signals reflected from the object 10 to generate an electrical reception signal.

The charging module 116 may charge the battery 114. The charging module 116 may receive power from the outside. According to an embodiment, the charging module 116 may receive power wirelessly. Furthermore, according to an embodiment, the charging module 116 may receive power by wire. The charging module 116 may transmit the received power to the battery 114.

The processor 118 may control the transmission module 113 to generate transmission signals to be applied to the respective transducers, considering the positions and focal points of the plurality of transducers.

The processor 118 may control the reception module 115 to generate ultrasound data, by performing analog-to-digital conversion on the reception signals received from the transducer 117, and summing the digitally converted reception signals, considering the positions and focal points of the plurality of transducers. According to an embodiment, when the probe 20 includes the image processor 130, an ultrasound image may be generated by using the generated ultrasound data.

When the probe 20 is implemented as a two-dimensional probe, the processor 118 may calculate a time delay value for digital beamforming for each sub-array, with respect to each of the plurality of sub-arrays included in the two-dimensional transducer array. Furthermore, the processor 118 may calculate a time delay value for analog beamforming with respect to each of transducers included in any one of the plurality of sub-arrays. The processor 118 may control an analog beamformer and a digital beamformer to generate transmission signals to be applied to the respective transducers, based on the time delay value for analog beamforming and the time delay value for digital beamforming. Furthermore, the processor 118 may control the analog beamformer to sum the signals received from the plurality of transducers, for each sub-array, based on the time delay value for analog beamforming. Furthermore, the processor 118 may control the ultrasound transceiving module 110 to perform analog-to-digital conversion on the signal summed for each sub-array. Furthermore, the processor 118 may control the digital beamformer to generate ultrasound data by summing the digitally converted signals based on the time delay value for digital beamforming.

The processor 118 may control the overall operation of the probe 20 and the operation of the components of the probe 20. The processor 118 may control various operations or functions of the probe 20 by executing programs or instructions stored in a memory 111. Furthermore, the processor 118 may control the operation of the probe 20 by receiving control signal from the input interface 109 of the probe 20 or the external device (e.g., the ultrasound imaging apparatus 40). Furthermore, the processor 118 may control the operation of the probe 20 by receiving a control signal from the input interface 109 or the external device. The input interface 109 may receive a user's input to control the probe 20. For example, the user's input may include an input for manipulating a button, a key pad, a mouse, a trackball, a jog switch, or a knob, an input for touching a touch pad or a touch screen, a voice input, a motion input, or a biometric information input (e.g., iris recognition or fingerprint recognition), but the disclosure is not limited thereto.

The display 112 may display an ultrasound image generated by the probe 20, an ultrasound image generated by processing ultrasound data generated by the probe 20, an ultrasound image received from the ultrasound imaging apparatus 40, or various pieces of information processed in the ultrasound imaging system 100. Furthermore, the display 112 may further display state information of the probe 20. The state information of the probe 20 may include at least one of device information of the probe 20, battery status information of the probe 20, frequency band information of the probe 20, output information of the probe 20, abnormality information of the probe 20, setting information of the probe 20, or temperature information of the probe 20.

The probe 20 may include one or a plurality of displays 112 based on the implementation type. Furthermore, the display 112 may include a touch panel or a touch screen. Furthermore, the display 112 may include a flexible display.

The communication module 119 may wirelessly transmit the generated ultrasound data or ultrasound image to the ultrasound imaging apparatus 40 through a wireless network. Furthermore, the communication module 119 may receive a control signal and data from the ultrasound imaging apparatus 40.

The ultrasound imaging apparatus 40 may receive ultrasound data or ultrasound images from the probe 20.

In an embodiment, when the probe 20 includes the image processor 130 capable of generating ultrasound images using ultrasound data, the probe 20 may transmit, to the ultrasound imaging apparatus 40, the ultrasound data or the ultrasound image generated by the image processor 130.

In an embodiment, when the probe 20 does not include the image processor 130 capable of generating ultrasound images using ultrasound data, the probe 20 may transmit the ultrasound data to the ultrasound imaging apparatus 40. The ultrasound data may include ultrasound raw data, and the ultrasound image may mean ultrasound image data.

The ultrasound imaging apparatus 40 may include the processor 120, the image processor 130, the display 140, the memory 150, the communication module 160, and the input interface 170.

The image processor 130 may generate or process an ultrasound image by using the ultrasound data received from the probe 20.

The display 140 may display, for example, the ultrasound images received from the probe 20, the ultrasound image generated by processing the ultrasound data received from the probe 20, or various pieces of information processed by the ultrasound imaging system 100. The ultrasound imaging apparatus 40 may include one or a plurality of displays 140 based on the implementation type. Furthermore, the display 140 may include a touch panel or a touch screen. Furthermore, the display 140 may include a flexible display.

The processor 120 may control the overall operation of the ultrasound imaging apparatus 40 and the operation of components of the ultrasound imaging apparatus 40. The processor 120 may perform or control various operations or functions of the ultrasound imaging apparatus 40 by executing the programs or applications stored in the memory 150. Furthermore, the processor 120 may control the operation of the ultrasound imaging apparatus 40 by receiving a control signal from the input interface 170 or the external device.

The ultrasound imaging apparatus 40 may include the communication module 160, and may be connected to an external device (e.g., the probe 20, a server, a medical device, or a portable device, such as a smartphone, a tablet PC, or a wearable device) to communicate therewith through the communication module 160.

The communication module 160 may include one or more components to enable communication with the external device. The communication module 160 may include at least one of, for example, a short-range communication module, a wired communication module, or a wireless communication module.

The communication module 160 of the ultrasound imaging apparatus 40 and the communication module 119 of the probe 20 may communicate with each other using a network or a short-range wireless communication method. For example, the communication module 160 of the ultrasound imaging apparatus 40 and the communication module 119 of the probe 20 may communicate with each other using any one of wireless data communication methods including a wireless LAN, Wi-Fi, Bluetooth, Zigbee, Wi-Fi direct (WFD), Infrared Data Association (IrDA), Bluetooth low energy (BLE), near field communication (NFC), wireless broadband Internet (Wibro), world interoperability for microwave access (WiMAX), shared wireless access protocol (SWAP), wireless gigabit alliance (WiGig), radio frequency (RF) communication, or 60 GHz millimeter wave (mm Wave) short-range communication.

To this end, the communication module 160 of the ultrasound imaging apparatus 40 and the communication module 119 of the probe 20 may include at least one of a wireless LAN communication module, a Wi-Fi communication module, a Bluetooth communication module, a Zigbee communication module, a WFD communication module, an IrDA module, a BLE communication module, an NFC communication module, a Wibro communication module, a WiMAX communication module, an SWAP communication module, a WiGig communication module, an RF communication module, or a 60 GHz mm Wave short-range communication module.

In an embodiment, the probe 20 may transmit the device information (e.g., ID information) of the probe 20 to the ultrasound imaging apparatus 40 by using a first communication method (e.g., BLE), and may be wirelessly paired with the ultrasound imaging apparatus 40. Furthermore, the probe 20 may transmit the ultrasound data and/or the ultrasound image to the ultrasound imaging apparatus 40 that is paired.

The device information of the probe 20 may include various pieces of information related to the serial number, model name, or battery status of the probe 20.

The ultrasound imaging apparatus 40 may receive the device information (e.g., ID information) of the probe 20 from the probe 20 by using the first communication method (e.g., BLE), and may be wirelessly paired with the probe 20. Furthermore, the ultrasound imaging apparatus 40 may transmit an activation signal to the probe 20 that is paired, and receive ultrasound data and/or ultrasound image from the probe 20. The activation signal may include a signal to control the operation of the probe 20.

In an embodiment, the probe 20 may transmit the device information (e.g., ID information) of the probe 20 to the ultrasound imaging apparatus 40 by using the first communication method (e.g., BLE), and may be wirelessly paired with the ultrasound imaging apparatus 40. Furthermore, the probe 20 may transmit ultrasound data and/or ultrasound images to the ultrasound imaging apparatus 40 that is paired by using the first communication method, by using a second communication method (e.g., 60 GHz millimeter wave or Wi-Fi).

The ultrasound imaging apparatus 40 may receive the device information (e.g., ID information) of the probe 20 from the probe 20 by using the first communication method (e.g., BLE), and may be wirelessly paired with the probe 20. Furthermore, the ultrasound imaging apparatus 40 may transmit an activation signal to the probe 20 that is paired, and receive the ultrasound data and/or ultrasound images from the probe 20 by using the second communication method (e.g., 60 GHz millimeter wave or Wi-Fi).

According to an embodiment, the first communication method used to pair the probe 20 and the ultrasound imaging apparatus 40 with each other may have a lower frequency band than the frequency band of second communication method used for the probe 20 to transmit the ultrasound data and/or ultrasound images to the ultrasound imaging apparatus 40.

The display 140 of the ultrasound imaging apparatus 40 may display a user interface (UI) indicating the device information of the probe 20. For example, the display 140 may display a UI indicating identification information of the probe 20, a pairing method between the ultrasound imaging apparatus 40 and the probe 20, a data communication status between the probe 20 and the ultrasound imaging apparatus 40, a data communication method between the ultrasound imaging apparatus 40 and the probe 20, or a battery status of the probe 20.

When the probe 20 includes the display 112, the display 112 of the probe 20 may display a UI indicating the device information of the probe 20. For example, the display 112 may display a UI indicating identification information of the probe 20, a pairing method between the ultrasound imaging apparatus 40 and the probe 20, a data communication status between the probe 20 and the ultrasound imaging apparatus 40, a data communication method between the ultrasound imaging apparatus 40 and the probe 20, or a battery status of the probe 20.

The communication module 160 may receive a control signal or data from the external device. The processor 120 may control the operation of the ultrasound imaging apparatus 40 based on the control signal received through the communication module 160.

Furthermore, the processor 120 may transmit a control signal to the external device through the communication module 160 to control the external device based on the transmitted control signal. The external device may operate based on the control signal received from the ultrasound imaging apparatus 40 or process data received from the ultrasound imaging apparatus 40.

The external device may receive or download programs or applications related to the ultrasound imaging apparatus 40 from the ultrasound imaging apparatus 40, the probe 20, or the server, and install the programs or applications in the external device to execute the installed programs or applications. The ultrasound imaging apparatus 40, the probe 20, or the server that provides the program or application may include a recording medium for storing instructions, commands, installation files, execution files, or relevant data of the program or application. The external device may be sold with the program or application installed.

The memory 150 may store various pieces of data or programs to drive and control the ultrasound imaging apparatus 40, input/output ultrasound data, or ultrasound images.

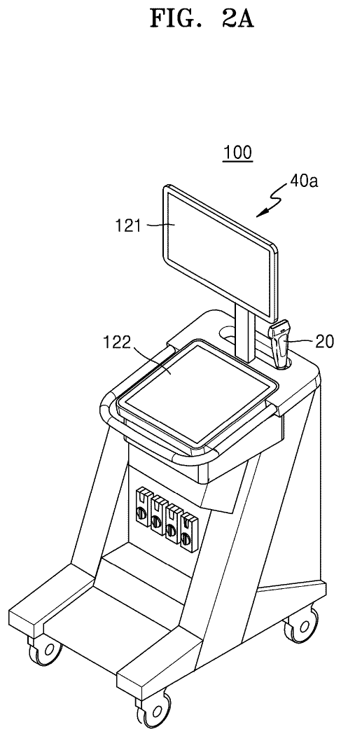

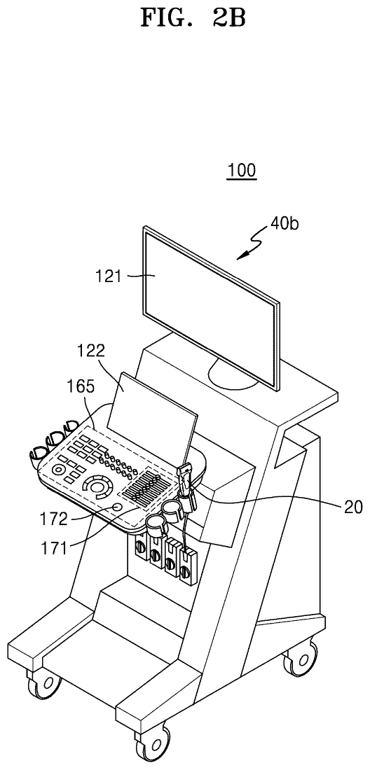

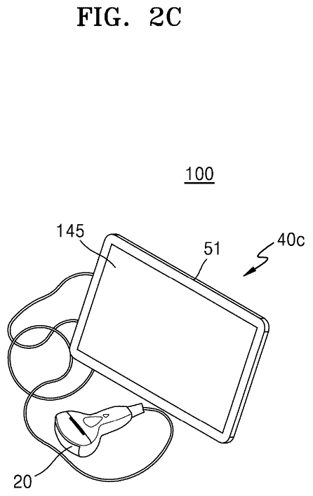

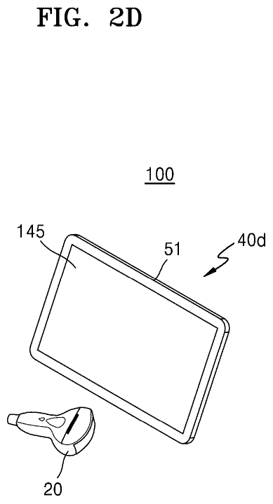

Examples of the ultrasound imaging system 100 according to an embodiment are described below with reference to FIGS. 2A, 2B, 2C, and 2D.

FIGS. 2A, 2B, 2C, and 2D are diagrams illustrating an ultrasound imaging apparatus according to an embodiment.

Referring to FIGS. 2A and 2B, ultrasound imaging apparatuses 40a and 40b may each include a main display 121 and a sub-display 122. The main display 121 and the sub-display 122 may correspond to the display 140 of FIGS. 1A and 1B. At least one of the main display 121 or the sub-display 122 may be implemented as a touchscreen. At least one of the main display 121 or the sub-display 122 may display an ultrasound image or various pieces of information processed in the ultrasound imaging apparatuses 40a and 40b. Furthermore, at least one of the main display 121 or the sub-display 122 may be implemented as a touch screen, and by providing a graphic user interface (GUI), may receive, from a user, data for controlling the ultrasound imaging apparatuses 40a and 40b. For example, the main display 121 may display an ultrasound image, and the sub-display 122 may display a control panel for controlling display of an ultrasound image in a GUI form. The sub-display 122 may receive data for controlling display of an image through the control panel displayed in the GUI form. For example, a time gain compensation (TGC) button, a lateral gain compensation (LGC) button, a freeze button, a trackball, a jog switch, or knob may be provided to the sub-display 122 as GUI.

The ultrasound imaging apparatuses 40a and 40b may each control display of an ultrasound image displayed on the main display 121 by using the received control data. Furthermore, the ultrasound imaging apparatuses 40a and 40b may be connected to the probe 20 by wire or wirelessly to transceive ultrasound signals with respect to an object.

Referring to FIG. 2B, the ultrasound imaging apparatus 40b may further include a control panel 165 in addition to the main display 121 and the sub-display 122. The control panel 165 may include a button, a trackball, a jog switch, or a knob, and receive, from the user, data for controlling the ultrasound imaging apparatus 40b. For example, the control panel 165 may include a TGC button 171 or a freeze button 172. The TGC button 171 may be a button for setting a TGC value for each depth of an ultrasound image. Furthermore, when an input of the freeze button 172 is detected while scanning an ultrasound image, the ultrasound imaging apparatus 40b may maintain a state of displaying a frame image at the corresponding time point, capture a frame image at the corresponding time point, or store a frame image at the corresponding time point.

The button, trackball, jog switch, or knob included in the control panel 165 may be provided in the main display 121 or the sub-display 122 as GUI. Furthermore, the ultrasound imaging apparatuses 40a and 40b may each be connected to the probe 20 to transceive ultrasound signals with respect to the object.

Furthermore, the ultrasound imaging apparatuses 40a and 40b may each include various forms of an input/output interface, such as a speaker, a light-emitting display (LED), or a vibration device. For example, the ultrasound imaging apparatuses 40a and 40b may each output various pieces of information in the form of graphics, sound, or vibration, through the input/output interface. Furthermore, the ultrasound imaging apparatuses 40a and 40b may output various notifications or data through the input/output interface.

Referring to FIGS. 2C and 2D, ultrasound imaging apparatuses 40c and 40d may each be implemented as a portable apparatus. Examples of portable ultrasound imaging apparatuses 40c and 40d may include a smart phone, a laptop computer, a PDA, or tablet PC, each including a probe and an application, but the disclosure is not limited thereto.

The ultrasound imaging apparatus 40c may include a main body 41. Referring to FIG. 2C, the probe 20 may be connected by wire to one side of the main body 41. To this end, the main body 41 may include a connection terminal having a detachable cable connected to the probe 20. The probe 20 may include a cable including a connection terminal that is connectable to the main body 41.

Referring to FIG. 2D, the probe 20 may be wirelessly connected to the ultrasound imaging apparatus 40d. The main body 41 may include an input/output interface (e.g., a touch screen). The in put/output interface may display an ultrasound image, various pieces of information processed in the ultrasound imaging apparatus, or GUI.

The ultrasound imaging apparatus 40d and the probe 20 may establish communication or may be paired by using a short-range wireless communication. For example, the ultrasound imaging apparatus 40d and the probe 20 may perform communication by using Bluetooth, BLE, Wi-Fi, or Wi-Fi direct.

The ultrasound imaging apparatuses 40c and 40d may control the probe 20 and output information related to the probe 20, by executing the program or application related to the probe 20. The ultrasound imaging apparatuses 40c and 40d may perform an operation related to the probe 20 while communicating with a certain server. The probe 20 may be registered on the ultrasound imaging apparatuses 40c and 40d or the certain server. The ultrasound imaging apparatuses 40c and 40d may each communicate with the probe 20 that is registered and may perform the operation related to the probe 20.

Furthermore, the ultrasound imaging apparatuses 40c and 40d may each include various forms of an input/output interface, such as a speaker, an LED, or a vibration device. For example, the ultrasound imaging apparatuses 40c and 40d may each output various pieces of information in the form of graphics, sound, or vibration through the input/output interface. Furthermore, the ultrasound imaging apparatuses 40c and 40d may each output various notifications or data through the input/output interface.

According to an embodiment, the ultrasound imaging apparatus 40a, 40b, 40c, or 40d may process an ultrasound image or obtain additional information from the ultrasound image by using an artificial intelligence (AI) model. According to an embodiment, the ultrasound imaging apparatus 40a, 40b, 40c, or 40d may use AI models to generate ultrasound images or perform processing, such as correction, image quality enhancement, encoding, or decoding, on the ultrasound images. Furthermore, according to an embodiment, the ultrasound imaging apparatus 40a, 40b, 40c, or 40d may use AI models to perform processing, such as defining reference lines, obtaining anatomical information, obtaining lesion information, extracting surfaces, defining boundaries, measuring length, measuring area, measuring volume, or generating annotations, from the ultrasound images.

The AI model may be provided on the ultrasound imaging apparatus 40a, 40b, 40c, or 40d or on a server.

The AI model may be implemented by using various artificial neural network models or a deep neural network. Furthermore, the AI model may be trained and generated by using various machine learning algorithms or deep learning algorithms. The AI model may be implemented by using a model, for example, a convolutional neural network (CNN), a recurrent neural network (RNN), a generative adversarial network (GAN), or a long short-term memory (LSTM).

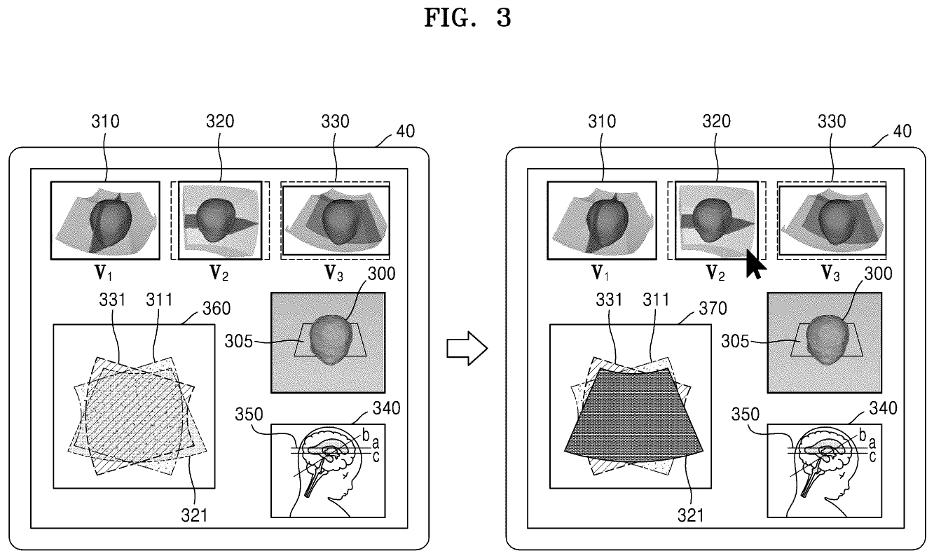

FIG. 3 is a diagram showing a method, performed by the ultrasound imaging apparatus 40, of displaying data from a merged volume, according to an embodiment.

Referring to FIG. 3, the ultrasound imaging apparatus 40 may generate a merged volume 300 by merging a plurality of original volumes. The ultrasound imaging apparatus 40 may provide not only a cross-sectional image 360 of the merged volume 300 indicating the cross-sectional area of the merged volume 300, but also a cross-sectional image 370 of the original volume corresponding to the cross-sectional area of the merged volume 300.

Referring to the left image in FIG. 3, the ultrasound imaging apparatus 40 may display the plurality of original volumes.

The ultrasound imaging apparatus 40 may obtain an original volume through a user's input to perform three-dimensional ultrasound scan on an object. For example, as illustrated in FIG. 3, the ultrasound imaging apparatus 40 may obtain a first original volume 310 by performing ultrasound scan on the head of a fetus in a first direction, a second original volume 320 by performing ultrasound scan in a second direction, and a third original volume 330 by performing ultrasound scan in a third direction. According to an embodiment, the ultrasound imaging apparatus 40 may obtain an original volume from an external device.

The ultrasound imaging apparatus 40 may generate the merged volume 300 by merging the obtained first to third original volumes 310, 320, and 330 based on a reference coordinate system. For example, the ultrasound imaging apparatus 40 may generate the merged volume 300 by merging the first original volume 310, the second original volume 320, and the third original volume 330 based on the coordinate system of the first original volume 310.

Volume merging may include generating one three-dimensional volume by transforming different volumes with respect to one coordinate system. Volume merging may include volume registration and volume stitching. In the specification, although merging between ultrasound volumes is disclosed, the embodiment of the disclosure may be applied to merging between volumes obtained from different modalities (e.g., an ultrasound volume, a magnetic resonance imaging (MRI) volume, or a computed tomography (CT) volume).

The volume registration may include transforming ultrasound volumes of the same object obtained at different time points and from different views, with respect to one coordinate system, to generate a three-dimensional volume for the object. The volume stitching may include stitching a plurality of volumes having partially overlapping scan areas together to generate a three-dimensional volume greater than the original volume.

The ultrasound imaging apparatus 40 may generate the merged volume 300 by combining data of the first to third original volumes 310, 320, and 330, which have been coordinate-transformed based on the reference coordinate system. For example, the ultrasound imaging apparatus 40 may select the highest intensity value or an average value for one coordinate, but the disclosure is not limited thereto.

Furthermore, the ultrasound imaging apparatus 40 may perform blending to minimize discontinuity of data when combining data of the first to third original volumes 310, 320, and 330. For example, the ultrasound imaging apparatus 40 may combine the data of the first to third original volumes 310, 320, and 330 by setting the contribution of original volumes for one coordinate differently based on alpha blending.

The ultrasound imaging apparatus 40 may display the merged volume 300 that is generated.

As the merged volume 300 is provided based on data imaged at various angles, the ultrasound imaging apparatus 40 may provide accurate information about a lesion of interest. Furthermore, as the ultrasound imaging apparatus 40 is capable of capturing only a limited field of sight in one scan, a relatively large area may be provided as one volume by stitching a plurality of ultrasound volumes. Accordingly, the ultrasound imaging apparatus 40 may accurately provide the overall structure of an object or the size of a lesion.

Furthermore, the ultrasound imaging apparatus 40 may provide the cross-sectional image 360 of the merged volume 300 indicating a cross-sectional area of interest 305 within the merged volume 300.

According to an embodiment, the ultrasound imaging apparatus 40 may identify predetermined structures within the merged volume 300, and determine the cross-sectional area of interest 305 within the merged volume 300 based on the identified structures. For example, the ultrasound imaging apparatus 40 may identify a brain structure, such as lateral ventricles or choroid plexus, within the merged volume 300 for the head of a fetus, and determine the coordinate value of the cross-sectional area of interest 305 to include the identified brain structure. Furthermore, for example, the ultrasound imaging apparatus 40 may identify a sagittal plane within the merged volume 300 for the head, and determine the identified sagittal plane as the cross-sectional area of interest 305.

According to an embodiment, the ultrasound imaging apparatus 40 may receive a user′s input to set the cross-sectional area of interest 305 within the merged volume 300. For example, the ultrasound imaging apparatus 40 may display a cross-sectional image 340 of the merged volume 300 for the head of a fetus or a cross-sectional image of an original volume, and receive a user′s input to set a position 350 of a cross-section of interest on the cross-sectional image 340 that is displayed.

The ultrasound imaging apparatus 40 may display the cross-sectional image 360 of the merged volume 300 indicating the cross-sectional area of interest 305 within the merged volume 300. The cross-sectional image 360 of the merged volume 300 may be an image obtained by combining an image 311 indicating the cross-sectional area of interest 305 within the first original volume 310, an image 321 indicating the cross-sectional area of interest 305 within the second original volume 320, and an image 331 indicating the cross-sectional area of interest 305 within the third original volume 330.

When ultrasound images are combined, a combined image may cause distortion of an object. For example, when a gradient difference between volume data for the same area is large, a blended image may distort an actual object. In particular, as ultrasound images have relatively high noise levels, lower contrast, and borderless properties than general images, deformations on original data such as blending are likely to cause object distortion. Distortion of objects in medical images may lead to diagnosis errors.

The ultrasound imaging apparatus 40 may prevent diagnosis errors due to distortion by providing the cross-sectional images of the first to third original volumes 310, 320, and 330 corresponding to the cross-sectional image of the merged volume 300.

Referring to the right image in FIG. 3, the ultrasound imaging apparatus 40 may display the image 321 of the second original volume 320 corresponding to the cross-sectional image 360 of the merged volume 300, based on the reception of a user′s input to select the second original volume 320.

For example, the ultrasound imaging apparatus 40 may obtain data of the cross-sectional area of interest 305 within the second original volume 320 transformed with respect to a reference coordinate system, based on the coordinate value of the cross-sectional area of interest 305. The ultrasound imaging apparatus 40 may generate the image 321 of the cross-sectional area of interest 305 within the second original volume 320 based on the obtained data.

Furthermore, for example, the ultrasound imaging apparatus 40 may generate a new cross-sectional image 370 of the merged volume 300 by combining data of the first original volume 310, the second original volume 320, and the third original volume 330 for the cross-sectional area of interest 305, and determining the data of the second original volume 320 as a value of an area for the area where the data of the selected second original volume 320 exists. The ultrasound imaging apparatus 40 may display the cross-sectional image 370 that is newly generated instead of the cross-sectional image 360 of the merged volume 300.

Accordingly, the user may check not only the cross-sectional image of the merged volume, but also the cross-sectional image of the original volume corresponding to the cross-sectional image of the merged volume.

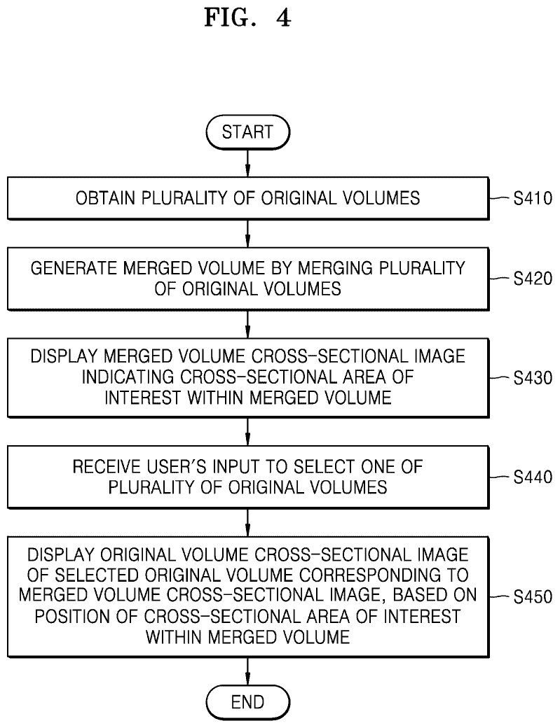

FIG. 4 is a flowchart of a method, performed by the ultrasound imaging apparatus 40, of displaying data from a merged volume, according to an embodiment.

In operation S410, the ultrasound imaging apparatus 40 may obtain a plurality of original volumes.

The ultrasound imaging apparatus 40 may receive a user′s input to select a menu for obtaining an ultrasound volume for an object. The ultrasound imaging apparatus 40 may receive an ultrasound echo signal for the object based on receiving a user's input to scan an object using a probe. The ultrasound imaging apparatus 40 may generate an ultrasound volume for an object based on the received ultrasound echo signal. An ultrasound volume may include three-dimensional coordinates and data at the coordinates.

In operation S420, the ultrasound imaging apparatus 40 may generate a merged volume by merging a plurality of original volumes.

Volume merging may include generating one three-dimensional volume by transforming different volumes with respect to one coordinate system. Volume merging may include volume registration and volume stitching.

For example, the ultrasound imaging apparatus 40 may receive a user′s input to set a reference volume from among the plurality of original volumes. The ultrasound imaging apparatus 40 may merge the plurality of original volumes based on the coordinate system of the reference volume.

The ultrasound imaging apparatus 40 may transform the coordinates of the remaining original volumes with respect to the coordinate system of the reference volume. The ultrasound imaging apparatus 40 may combine data of the reference volume and data of the coordinate-transformed original volumes. The ultrasound imaging apparatus 40 may perform blending when combining data of the original volumes.

In operation S430, the ultrasound imaging apparatus 40 may display a merged volume cross-section image indicating a cross-sectional area of interest within the merged volume.

The ultrasound imaging apparatus 40 may receive a user's input to set the cross-sectional area of interest within the merged volume. The ultrasound imaging apparatus 40 may determine a cross-sectional area of interest based on predetermined structures within the merged volume.

The cross-sectional area of interest may include a plurality of cross-sectional areas of interest. For example, the cross-sectional area of interest may include a plane A, a plane B, and a plane C of the merged volume. Furthermore, for example, the cross-sectional area of interest may include a plurality of predetermined cross-sectional areas of interest within the merged volume indicating the brain of a fetus.

The ultrasound imaging apparatus 40 may display a plurality of merged volume cross-section images. The ultrasound imaging apparatus 40 may receive a user's input to select one of the plurality of merged volume cross-section images that are displayed.

In operation S440, the ultrasound imaging apparatus 40 may receive a user's input to select one of the plurality of original volumes.

The ultrasound imaging apparatus 40 may receive a user's input to select one of the plurality of original volumes corresponding to the merged volume cross-section image.

The ultrasound imaging apparatus 40 may display a user interface for selecting one of the plurality of original volumes. The user interface may include the cross-sectional images of a plurality of original volumes as identification information of the plurality of original volumes.

In operation S450, the ultrasound imaging apparatus 40 may display an original volume cross-section image of a selected original volume corresponding to the merged volume cross-section image, based on the position of the cross-sectional area of interest within the merged volume.

The original volume cross-section image may include data of a selected original volume merged into a merge volume cross-section image.

The ultrasound imaging apparatus 40 may obtain data of the cross-sectional area of interest of a reference coordinate system as an original volume cross-section image corresponding to the merged volume cross-section image, among the data within the original volume arranged based on a reference coordinate system. The ultrasound imaging apparatus 40 may display the obtained original volume cross-section image.

The ultrasound imaging apparatus 40 may generate a new merged volume cross-section image so as to preferentially display data of the selected original volume for an area where the data of the original volume selected from the cross-sectional area of interest within the merged volume overlaps data of the other original volumes. The ultrasound imaging apparatus 40 may display a newly generated merged volume cross-section image.

The ultrasound imaging apparatus 40 may display identification information of the selected original volume with the original volume cross-section image.

According to an embodiment, the ultrasound imaging apparatus 40 may receive a user′s input to set the priority of the plurality of original volumes. The ultrasound imaging apparatus 40 may generate a new merged volume cross-section image for the area where the data of the plurality of original volumes overlap so as to preferentially display data of an original volume with relatively high priority. The ultrasound imaging apparatus 40 may display a newly generated merged volume cross-section image.

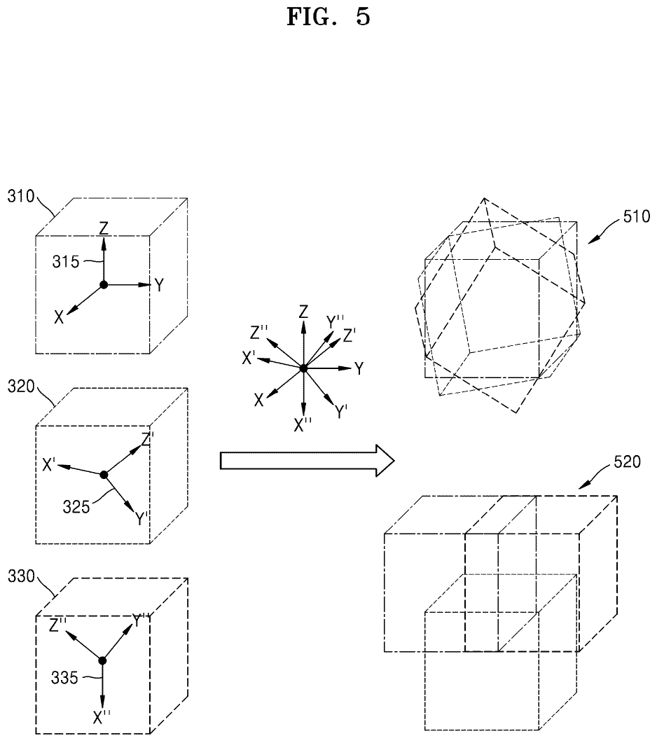

FIG. 5 is a diagram showing a method, performed by the ultrasound imaging apparatus 40, of registering or stitching original volumes, according to an embodiment.

Referring to FIG. 5, when the first to third original volumes 310, 320, and 330 are ultrasound volumes for the same object obtained from different views, the ultrasound imaging apparatus 40 may generate a registered volume 510 by registering the first to third original volumes 310, 320, and 330. Furthermore, the first to third original volumes 310, 320, and 330 are a plurality of volumes having partially overlapping scan areas for the object, the ultrasound imaging apparatus 40 may generate a stitched volume 520 by stitching the first to third original volumes 310, 320, and 330.

A coordinate system 315 of the first original volume 310, a coordinate system 325 of the second original volume 320, and a coordinate system 335 of the third original volume 330 may be different from one another. In order to merge the first to third original volumes 310, 320, and 330, the ultrasound imaging apparatus 40 may convert coordinate values of the first to third original volumes 310, 320, and 330 based on one coordinate system. For example, the ultrasound imaging apparatus 40 may convert coordinate values of voxel data of the second original volume 320 and the third original volume 330 based on the coordinate system 315 of the first original volume 310.

The ultrasound imaging apparatus 40 may transform the first to third original volumes 310, 320, and 330 with respect to one coordinate system based on various methods. For example, the ultrasound imaging apparatus 40 may extract, from volume data, anatomical features (e.g., blood vessels or a fetus's head outline), and transform the first to third original volumes 310, 320, and 330 with respect to one coordinate system (e.g., rigid body conversion) to match the extracted anatomical features. Furthermore, for example, the ultrasound imaging apparatus 40 may transform the first to third original volumes 310, 320, and 330 with respect to one coordinate system to match intensity values of voxels of the original volumes as much as possible, based on the intensity of voxels within a volume. Furthermore, for example, the ultrasound imaging apparatus 40 may transform the first to third original volumes 310, 320, and 330 with respect to one coordinate system by using an artificial intelligence model that outputs a merged volume when a plurality of volumes are input.

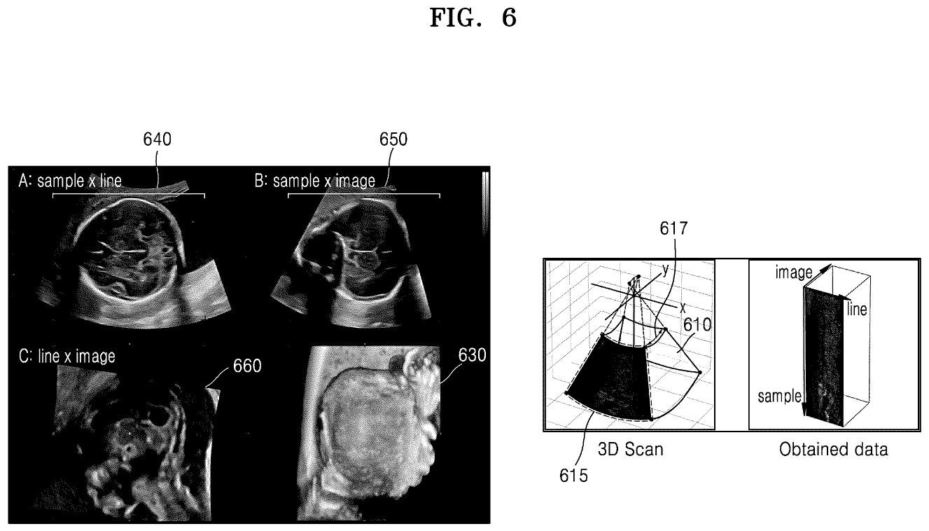

FIG. 6 is a diagram showing a method, performed by the ultrasound imaging apparatus 40, of displaying cross-sections A, B, and C of an ultrasound volume, according to an embodiment.

Referring to FIG. 6, the ultrasound imaging apparatus 40 may display an A cross-section image 640, a B cross-section image 650, and a C cross-section image 660 of an ultrasound volume 630.

The ultrasound imaging apparatus 40 may emit an ultrasound signal to a cross-sectional area 615 of object through a probe, and receive an ultrasound echo signal from the cross-sectional area 615. Furthermore, the ultrasound imaging apparatus 40 may receive ultrasound echo signals with respect to a three-dimensional area 610 by performing scanning on cross-sectional areas neighboring in a scan direction 617. The ultrasound imaging apparatus 40 may simultaneously scan the three-dimensional area 610 depending on the type of a probe.

The ultrasound imaging apparatus 40 may determine coordinates for the three-dimensional area 610 and data at the coordinates based on the ultrasound echo signal with respect to the three-dimensional area 610.

In this case, the direction in which the ultrasound signal is irradiated may be referred to as a sample direction, a direction in which transducers are arranged may be referred to as a line direction, and the scan direction 617 may be referred to as an image direction.

The ultrasound imaging apparatus 40 may display a cross-sectional image formed by the sample direction and the line direction as the A cross-section image 640, a cross-sectional image formed by the sample direction and the image direction as the B cross-section image 650, and a cross-sectional image formed by the line direction and the image direction as the C cross-section image 660.

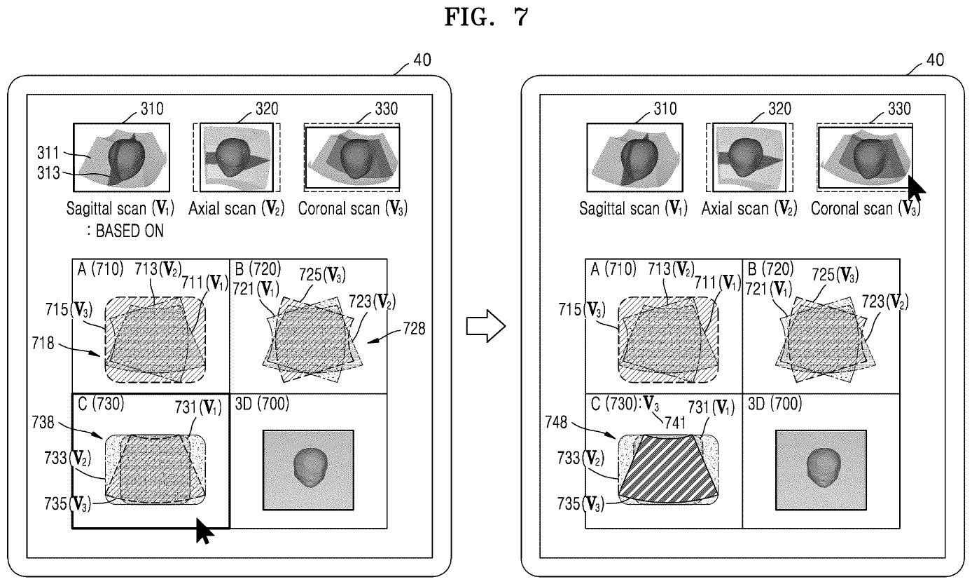

FIG. 7 is a diagram showing a method, performed by the ultrasound imaging apparatus 40, of displaying a cross-sectional image of a registered volume, according to an embodiment.

Referring to FIG. 7, the ultrasound imaging apparatus 40 may display an A cross-section image 718, a B cross-section image 728, and a C cross-section image 738 of a registered volume 700.

As illustrated in FIG. 7, by placing a probe in a vertical direction that divides the head of a fetus into the left and right sections, above the face of the fetus, and receiving a user′s input to scan the head of the fetus to the left and right sides, the ultrasound imaging apparatus 40 may obtain a sagittal scan volume (V₁, 310). Furthermore, by placing the probe in a direction that divides the head of the head of the fetus into the upper and lower sections, above the face of the fetus, and receiving a user's input to scan the head of the fetus to the upper and lower sides, the ultrasound imaging apparatus 40 may obtain an axial scan volume (V₂, 320). Furthermore, by placing the probe in a direction that divides the head of the fetus into the front and back sections, beside the head of the fetus, and receiving a user′s input to scan the head of the fetus to the front and back sides, the ultrasound imaging apparatus 40 may obtain a coronal scan volume (V₃, 330).

Although FIG. 7 illustrates volumes obtained through the sagittal scan, the axial scan, and the coronal scan, this is an embodiment for convenience of explanation, and a method of scanning for an original volume is not limited thereto. Furthermore, the number and type of original volumes for a merged volume and original volume are not limited. For example, the ultrasound imaging apparatus 40 may generate a merged volume by merging a first sagittal scan volume and a second sagittal scan volume.

As illustrated in FIG. 7, when displaying the first original volume 310, the ultrasound imaging apparatus 40 may display the image 311 indicating the scan area of a probe and the image 313 indicating the direction in which a probe is arranged. Furthermore, although not illustrated in FIG. 7, the ultrasound imaging apparatus 40 may display an image indicating the position of a probe and the scan direction of the probe, based on the first original volume 310.

The ultrasound imaging apparatus 40 may generate the registered volume 700 by registering the sagittal scan volume 310, the axial scan volume 320, and the coronal scan volume 330 based on the coordinate system of the sagittal scan volume 310.

The A cross-section image 718 of the registered volume 700 may be an image obtained by merging an A cross-section image 711 of the sagittal scan volume 310, a B cross-section image 713 of the axial scan volume 320, and a C cross-section image 715 of the coronal scan volume 330.

The B cross-section image 728 of the registered volume 700 may be an image obtained by merging a B cross-section image 721 of the sagittal scan volume 310, an A cross-section image 723 of the axial scan volume 320, and a B cross-section image 725 of the coronal scan volume 330.

Furthermore, the C cross-section image 738 of the registered volume 700 may be an image obtained by merging a C cross-section image 731 of the sagittal scan volume 310, a C cross-section image 733 of the axial scan volume 320, and an A cross-section image 735 of the coronal scan volume 330.

The ultrasound imaging apparatus 40 may receive a user's input to display an original volume cross-section image corresponding to a registered volume cross-section image 718, 728, or 738.

For example, the ultrasound imaging apparatus 40 may select a C cross-section display area 730 of the registered volume 700, and receive a user's input to select the coronal scan volume 330.

Referring to the right image in FIG. 7, the ultrasound imaging apparatus 40 may display an A cross-section image 735 of the coronal scan volume 330 in the C cross-section display area 730, based on the reception of the user's input. The A cross-section image 735 of the coronal scan volume 330 may be an image indicating the cross-sectional area of the coronal scan volume 330 corresponding to the C cross-section area of the registered volume 700.

According to an embodiment, the ultrasound imaging apparatus 40 may display the A cross-section image 735 of the coronal scan volume 330.

According to an embodiment, the ultrasound imaging apparatus 40 may display a new C cross-section image 748 in which the A cross-section image 735 of the coronal scan volume 330 is displayed prior to data of the other original volumes 310 and 320.

According to an embodiment, the ultrasound imaging apparatus 40 may display, in the C cross-section display area 730, information 741 indicating that a cross-sectional image of the coronal scan volume 330 is displayed.

According to an embodiment, based on the reception of the user's input to select the axial scan volume 320, the ultrasound imaging apparatus 40 may display the C cross-section image 733 of the axial scan volume 320 in the C cross-section display area 730.

According to an embodiment, based on the reception of the user's input to select the registered volume 700 again, the ultrasound imaging apparatus 40 may display again the C cross-section image 738 of the registered volume 700.

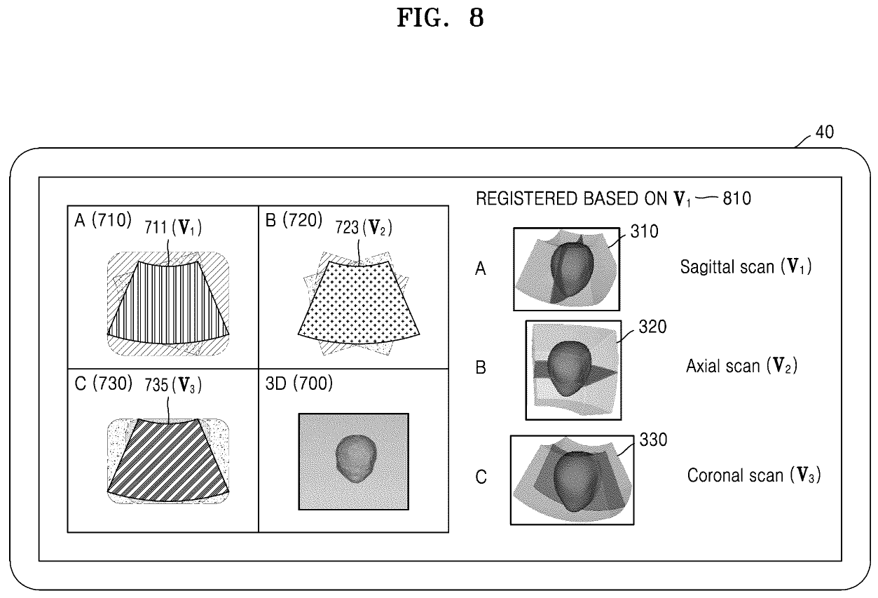

FIG. 8 is a diagram showing a method, performed by the ultrasound imaging apparatus 40, of displaying a cross-sectional image of a merged volume, according to an embodiment.

Referring to FIG. 8, the ultrasound imaging apparatus 40 may display the cross-sectional images of the first to third original volumes 310, 320, and 330 corresponding to a cross-section A, a cross-section B, and a cross-section C of the registered volume 700.

The ultrasound imaging apparatus 40 may generate the registered volume 700 by registering the sagittal scan volume 310, the axial scan volume 320, and the coronal scan volume 330 based on the coordinate system of the sagittal scan volume 310.

The ultrasound imaging apparatus 40 may display information 810 indicating that registration has been made based on the coordinate system of the sagittal scan volume 310.

The ultrasound imaging apparatus 40 may obtain the A cross-section image 711 of the sagittal scan volume 310 corresponding to the cross-section A of the registered volume 700, based on the selecting of the A cross-section display area 710 of the registered volume 700 and the reception of the user's input to select the sagittal scan volume 310. The ultrasound imaging apparatus 40 may display the A cross-section image 711 of the sagittal scan volume 310 in the A cross-section display area 710.

The ultrasound imaging apparatus 40 may obtain the A cross-section image 723 of the axial scan volume 320 corresponding to the cross-section B of the registered volume 700, based on the selecting of the B cross-section display area 720 of the registered volume 700 and the reception of the user's input to select the axial scan volume 320. The ultrasound imaging apparatus 40 may display the A cross-section image 723 of the axial scan volume 320 in the B cross-section display area 720.

The ultrasound imaging apparatus 40 may obtain the A cross-section image 735 of the coronal scan volume 330 corresponding to the cross-section C of the registered volume 700, based on the selecting of the C cross-section display area 730 of the registered volume 700 and the reception of the user's input to select the coronal scan volume 330. The ultrasound imaging apparatus 40 may display the A cross-section image 735 of the coronal scan volume 330 in the C cross-section display area 730.

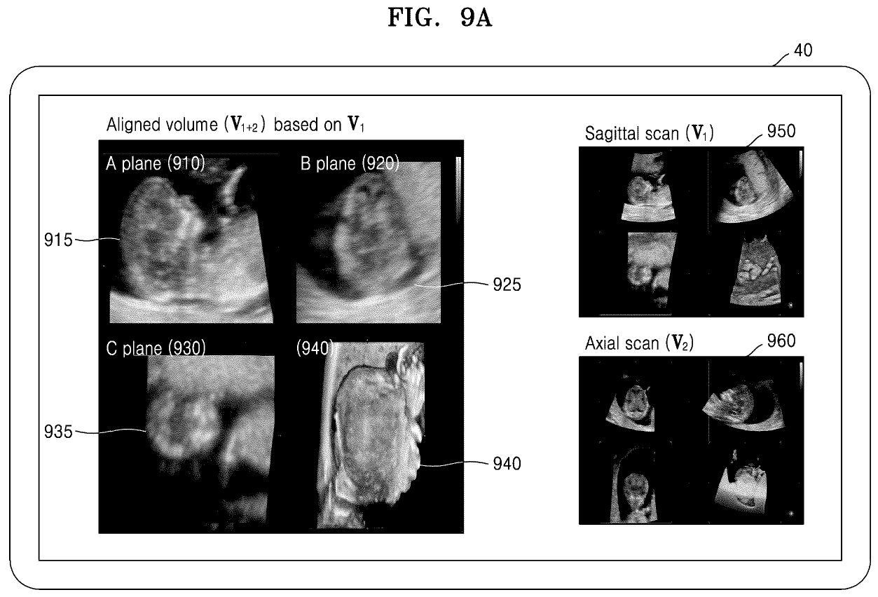

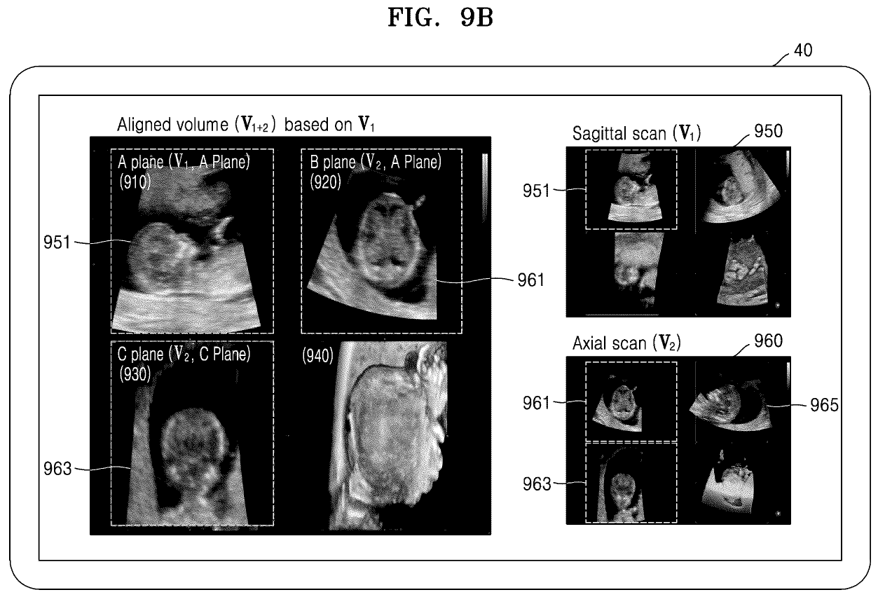

FIGS. 9A and 9B are diagrams showing a method, performed by the ultrasound imaging apparatus 40, of providing a user interface for selecting a cross-sectional image of an original volume, according to an embodiment.

Referring to FIG. 9A, the ultrasound imaging apparatus 40 may display cross-section images of an original volumes as a selection option of the original volume.

The ultrasound imaging apparatus 40 may generate a registered volume 940 by registering the sagittal scan volume 310 of FIG. 8 and the axial scan volume 320 of FIG. 8 based on the coordinate system of the sagittal scan volume 310 of FIG. 8.

The ultrasound imaging apparatus 40 may display the registered volume 940 and an A cross-section image 915, a B cross-section image 925, and a C cross-section image 935 of the registered volume 940.

The ultrasound imaging apparatus 40 may display the cross-sectional images of an original volume as icons for selecting the original volume. Referring to FIG. 9A, the ultrasound imaging apparatus 40 may display, as a selection icon for the sagittal scan volume 310 of FIG. 8, a thumbnail image 950 including a rendering image of the sagittal scan volume 310 of FIG. 8, the A cross-section image, the B cross-section image, and the C cross-section image. Furthermore, the ultrasound imaging apparatus 40 may display, as selection icons for the axial scan volume 320 of FIG. 8, a thumbnail image 960 including a rendering image of the axial scan volume 320 of FIG. 8 and the A cross-section image, the B cross-section image, and the C cross-section image.

Referring to FIG. 9B, the ultrasound imaging apparatus 40 may display an A cross-section image 951 of the sagittal scan volume 310 of FIG. 8 in an A cross-section display area 910, based on the selecting of the A cross-section display area 910 of the registered volume 940 and the reception of the user′s input to select the thumbnail image 950 of the sagittal scan volume 310 of FIG. 8. Furthermore, the ultrasound imaging apparatus 40 may display information indicating that a cross-sectional image displayed in the A cross-section display area 910 is the A cross-section image 951 of the sagittal scan volume 310 of FIG. 8.

Furthermore, the ultrasound imaging apparatus 40 may display an A cross-section image 961 of the axial scan volume 320 of FIG. 8 in a B cross-section display area 920, based on the selecting of the B cross-section display area 920 of the registered volume 940 and the reception of the user's input to select the thumbnail image 960 of the axial scan volume 320 of FIG. 8.

Furthermore, the ultrasound imaging apparatus 40 may display a C cross-section image 963 of the axial scan volume 320 of FIG. 8 in a C cross-section display area 930, based on the selecting of the C cross-section display area 930 of the registered volume 940 and the reception of the user's input to select the thumbnail image 960 of the axial scan volume 320 of FIG. 8.

According to an embodiment, the ultrasound imaging apparatus 40 may display the cross-sectional images of the original volumes corresponding to the selected cross-section display area 910, 920 or 930 by distinguishing the cross-sectional images of the original volumes from the other cross-sectional images, based on the reception of the user's input to select the cross-section display area 910, 920 or 930 of the registered volume 940. For example, the ultrasound imaging apparatus 40 may display the A cross-section image 951 of the sagittal scan volume 310 of FIG. 8 corresponding to the A cross-section image of the registered volume 940 and the B cross-section image 965 of the axial scan volume 320 of FIG. 8 by distinguishing the cross-sectional images of the original volumes from the other cross-sectional images, based on the reception of the user′s input to select the A cross-section display area 910. Accordingly, the user may easily distinguish the cross-sectional images of the original volume corresponding to the A cross-section image of the registered volume.

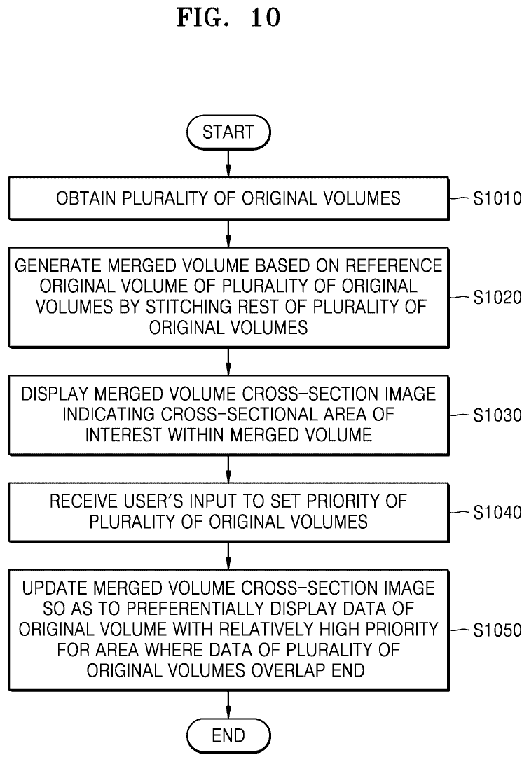

FIG. 10 is a flowchart showing a method, performed by the ultrasound imaging apparatus 40, of displaying a cross-sectional image of a merged volume based on priorities of original volumes, according to an embodiment.

In operation S1010, the ultrasound imaging apparatus 40 may obtain a plurality of original volumes. The operation S1010 may be described with reference to the operation S410 of FIG. 4.

In operation S1020, the ultrasound imaging apparatus 40 may generate a merged volume based on a reference original volume of the plurality of original volumes by stitching the rest of a plurality of original volumes.

The plurality of original volumes may be a plurality of volumes having partially overlapping scan areas. The ultrasound imaging apparatus 40 may generate a three-dimensional volume greater than the original volume by stitching together a plurality of original volumes.

The ultrasound imaging apparatus 40 may determine a coordinate system of the reference original volume of the plurality of original volumes as a reference coordinate system, and convert coordinate values of the other original volumes based on the reference coordinate system. The ultrasound imaging apparatus 40 may generate a stitched volume by combining data of the original volumes for the area where the data of the original volumes overlap, among voxels of the reference coordinate system.

In operation S1030, the ultrasound imaging apparatus 40 may display a merged volume cross-section image indicating a cross-sectional area of interest within the merged volume. The operation S1030 may be described with reference to the operation S430 of FIG. 4.

In operation S1040, the ultrasound imaging apparatus 40 may receive a user's input to set the priority of the plurality of original volumes.

The ultrasound imaging apparatus 40 may receive a user's input to set one original volume to have relatively high priority than the other original volumes. The ultrasound imaging apparatus 40 may receive a user's input to set the priority of each original volume differently.

In operation S1050, the ultrasound imaging apparatus 40 may update the merged volume cross-section image so as to preferentially display data of an original volume with relatively high priority for the area where data of the plurality of original volumes overlap.

The ultrasound imaging apparatus 40 may generate a new merged volume cross-section image for a cross-sectional area of interest, so as to preferentially display data of an original volume with relatively high priority for the area where the data of the original volumes overlap. The ultrasound imaging apparatus 40 may display a newly generated merged volume cross-section image.

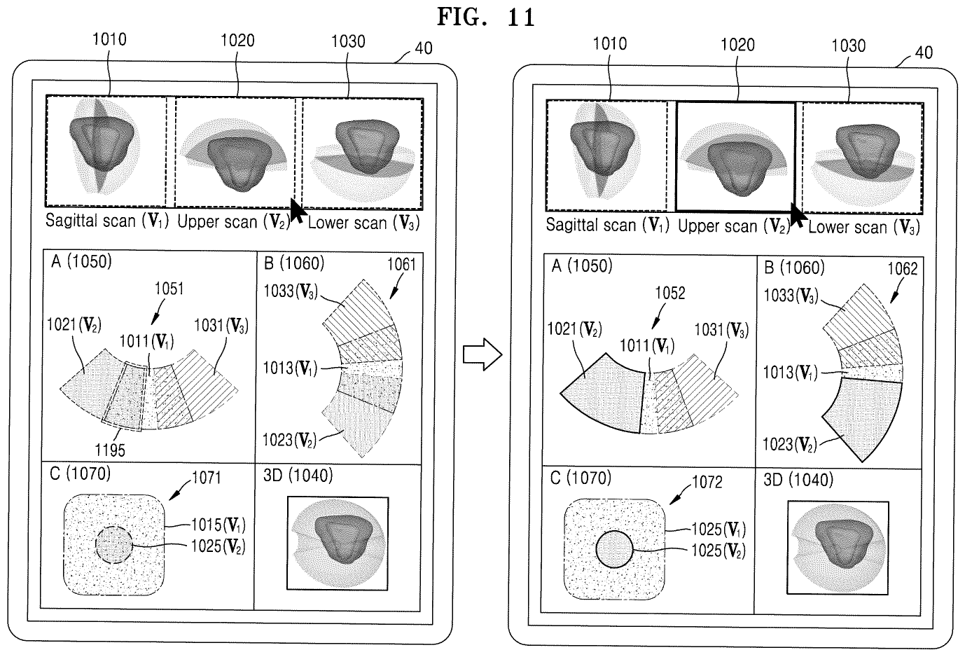

FIG. 11 is a diagram showing a method, performed by the ultrasound imaging apparatus 40, of displaying cross-sections A, B, and C of a stitched volume, according to an embodiment.

Referring to FIG. 11, the ultrasound imaging apparatus 40 may generate a stitched volume 1040 by stitching a first original volume 1010, a second original volume 1020, and a third original volume 1030 based on the coordinate system of the first original volume 1010.

The first original volume 1010 may be an ultrasound volume scanned to the left and right with respect to the sagittal plane of an area of interest. The second original volume 1020 may be a volume scanned to include an upper portion of the area of interest. The third original volume 1030 may be a volume scanned to include a lower portion of the area of interest.

The ultrasound imaging apparatus 40 may display an A cross-section image 1051 of the stitched volume 1040 in an A cross-section display area 1050. The A cross-section image 1051 of the stitched volume 1040 may be an image obtained by stitching an image 1011 of an A cross-section area of the first original volume 1010, an image 1021 of the A cross-section area in the second original volume 1020 that is coordinate-transformed, and an image 1031 of the A cross-section area in the third original volume 1030 that is coordinate-transformed.

Furthermore, the ultrasound imaging apparatus 40 may display a B cross-section image 1061 of the stitched volume 1040 in a B cross-section display area 1060. The B cross-section image 1061 of the stitched volume 1040 may be an image obtained by stitching an image 1013 of a B cross-section area of the first original volume 1010, an image 1023 of the B cross-section area in the second original volume 1020 that is coordinate-transformed, and an image 1033 of the B cross-section area in the third original volume 1030 that is coordinate-transformed.

Furthermore, the ultrasound imaging apparatus 40 may display a C cross-section image 1071 of the stitched volume 1040 in a C cross-section display area 1070. The C cross-section image 1071 of the stitched volume 1040 may be an image obtained by stitching an image 1015 of a C cross-section area of the first original volume 1010 and an image 1025 of the C cross-section area in the second original volume 1020 that is coordinate-transformed.

As the first original volume 1010, the second original volume 1020, and the third original volume 1030 are volumes scanned such that only portions of the scan area overlap each other, as illustrated in FIG. 11, only portions of the cross-sectional images of an original volume may overlap. For example, referring to the left image in FIG. 11, the A cross-section image 1051 of the stitched volume 1040 may include an overlapping area 1195 in which the image 1011 of the A cross-section area of the first original volume 1010 overlaps the image 1021 of the A cross-section area in the second original volume 1020 that is coordinate-transformed.

The ultrasound imaging apparatus 40 may receive a user's input to select one of the plurality of original volumes. For example, the ultrasound imaging apparatus 40 may receive a user′s input to select the second original volume 1020 from among a plurality of original volumes.

Referring to the right image in FIG. 11, the ultrasound imaging apparatus 40 may display cross-section images 1052, 1062, and 1072 of the stitched volume 1040 so as to preferentially display data of the second original volume 1020 that is selected, in an area where data of the original volumes overlap.

For example, the ultrasound imaging apparatus 40 may generate a new A cross-section image 1052 to display data of the second original volume 1020 that is selected for the area where the image 1011 of the first original volume 1010 overlaps the image 1021 of the second original volume 1020, in the A cross-section image 1051 of the stitched volume 1040. The ultrasound imaging apparatus 40 may display the new A cross-section image 1052 that is generated, in the A cross-section display area 1050.

According to an embodiment, the ultrasound imaging apparatus 40 may display an indicator indicating an area corresponding to the selected original volume so as to identify an area corresponding to the selected original volume on the cross-section images 1052, 1062, and 1072 of the stitched volume 1040. For example, referring to the right image in FIG. 11, according to the reception of a user's input to select the second original volume 1020, edges of the areas 1021, 1023, and 1025 corresponding to the selected second original volume may be emphasized on the cross-section images 1052, 1062, and 1072 of the stitched volume 1040.

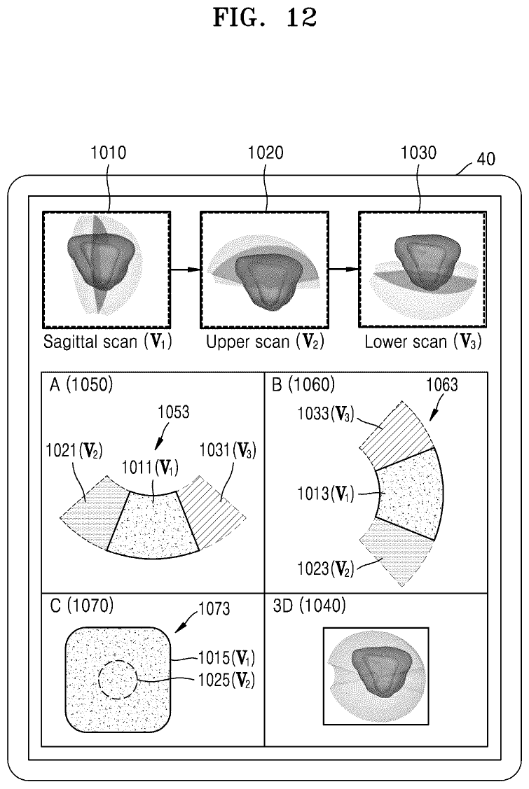

FIG. 12 is a diagram showing a method, performed by the ultrasound imaging apparatus 40, of displaying a cross-sectional image of a stitched volume based on priorities of original volumes, according to an embodiment.

Referring to FIG. 12, the ultrasound imaging apparatus 40 may receive a user's input to set priorities of the first, second, and third original volumes 1010, 1020, and 1030. The ultrasound imaging apparatus 40 may preferentially display data of the original volume with high priority for the area where data of the original volume overlap, based on the priorities of the first, second, and third original volumes 1010, 1020, and 1030.

For example, referring to FIG. 12, the ultrasound imaging apparatus 40 may receive a user's input to set priorities in the order of the first original volume 1010, the second original volume 1020, and the third original volume 1030. The ultrasound imaging apparatus 40 may display data of the first original volume 1010 for an area where the first original volume 1010 overlaps the second original volume 1020, based on the reception of the user's input to set priorities, and display data of the second original volume 1020 for an area where the second original volume 1020 overlaps the third original volume 1030. Furthermore, the ultrasound imaging apparatus 40 may display data of the first original volume 1010 for an area where the first original volume 1010 overlaps the third original volume 1030.

The ultrasound imaging apparatus 40 may generate an A cross-section image 1053, a B cross-section image 1063, and a C cross-section image 1073 based on the set priorities. The ultrasound imaging apparatus 40 may display the A cross-section image 1053, the B cross-section image 1063, and the C cross-section image 1073 that are generated.

As illustrated in FIG. 12, there may be an effect that a cross-sectional image of an original volume with relatively high priority is displayed in front and a cross-sectional image of an original volume with relatively low priority is hidden behind.

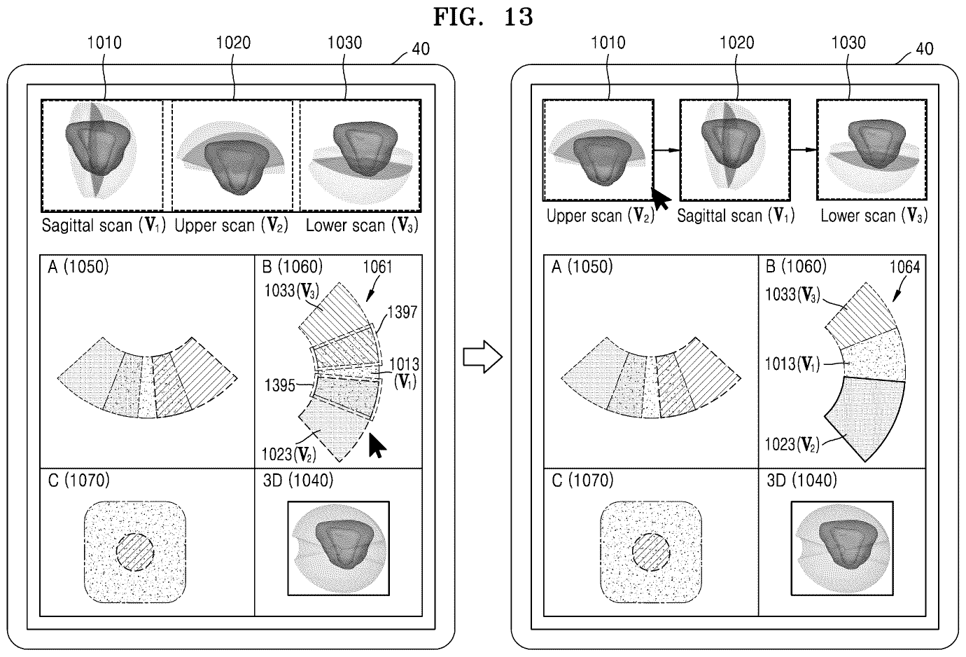

FIG. 13 is a diagram showing a method, performed by the ultrasound imaging apparatus 40, of selecting the priorities of original volumes for one cross-sectional image, according to an embodiment.

Referring to FIG. 13, the ultrasound imaging apparatus 40 may receive a user′s input to set the priorities of original volumes for one cross-sectional image.

Referring to the left image in FIG. 13, the ultrasound imaging apparatus 40 may receive a user's input to select the B cross-section image 1061 of the stitched volume 1040.

Referring to the right image in FIG. 13, the ultrasound imaging apparatus 40 may receive a user's input to set priority in the order of the second original volume 1020, the first original volume 1010, and the third original volume 1030 for the B cross-section image 1061 that is selected. based on the reception of the user's input to set priorities, the ultrasound imaging apparatus 40 may generate a new B cross-section image 1064 of the stitched volume 1040 by selecting data of the second original volume 1020 for an area 1395 where the first original volume 1010 overlaps the second original volume 1020 and selecting data of the first original volume 1010 for an area 1397 where the first original volume 1010 overlaps the third original volume 1030.

The ultrasound imaging apparatus 40 may display the B cross-section image 1064 that is generated, as the B cross-section image of the stitched volume 1040, in the B cross-section display area 1060.

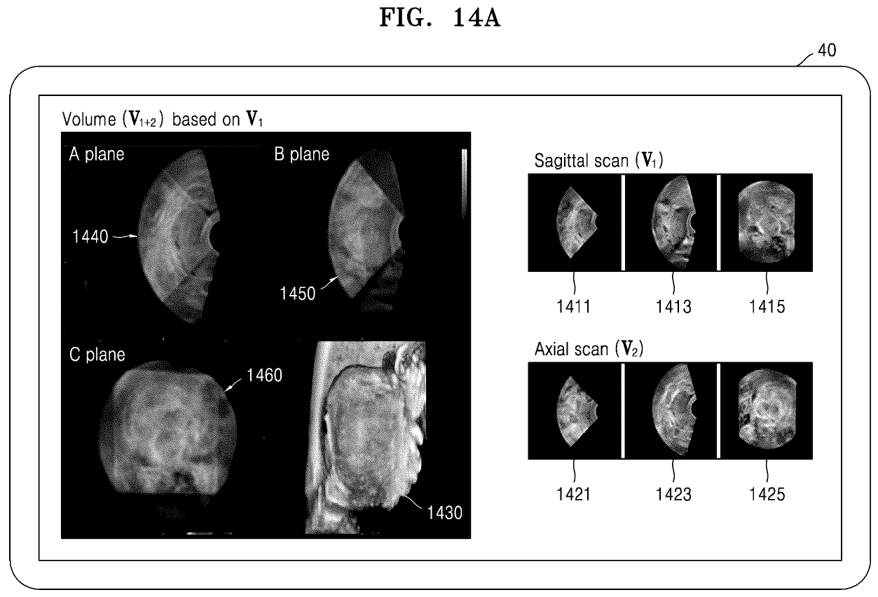

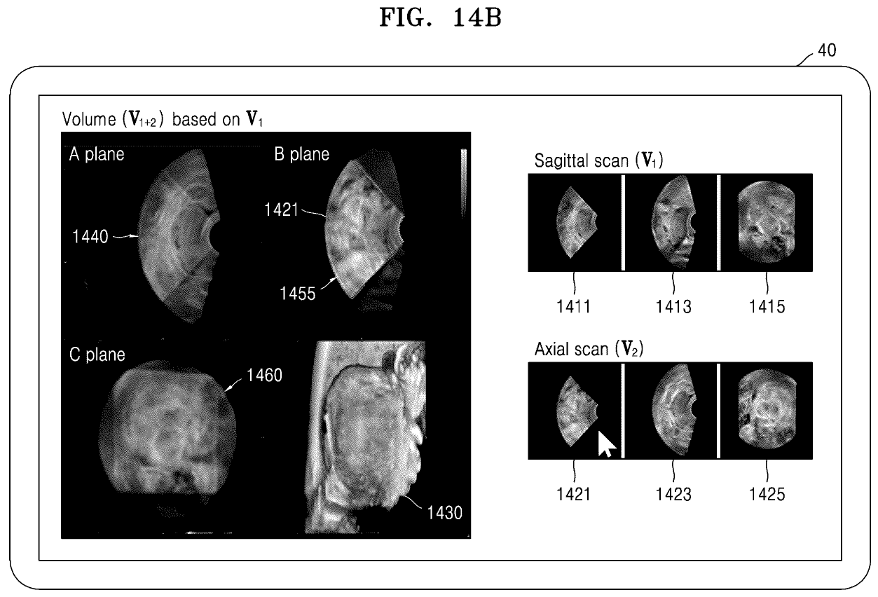

FIGS. 14A and 14B are diagrams showing a method, performed by the ultrasound imaging apparatus 40, of displaying a cross-sectional image of a merged volume, according to an embodiment.

Referring to FIG. 14A, the ultrasound imaging apparatus 40 may generate the merged volume 1430 by merging a first original volume V₁ and a second original volume V₂ based on the coordinate system of the first original volume V₁. The ultrasound imaging apparatus 40 may display an A cross-section image 1440, a B cross-section image 1450, and a C cross-section image 1460 of the merged volume 1430.

The first original volume V₁ may be a volume obtained through the sagittal scan of an object, and the second original volume V₂ may be a volume obtained through the axial scan of the object.

Furthermore, the ultrasound imaging apparatus 40 may display an A cross-section image 1441, a B cross-section image 1413, and a C cross-section image 1415 of the first original volume V₁, as selection icons. Furthermore, the ultrasound imaging apparatus 40 may display an A cross-section image 1421, a B cross-section image 1423, and a C cross-section image 1425 of the second original volume V₂, as selection icons. According to an embodiment, as illustrated in FIG. 14A, the ultrasound imaging apparatus 40 may display the cross-sectional images of an original volume in the form of thumbnails.

The A cross-section image 1440 of the merged volume 1430 may be generated by merging the A cross-section image 1441 of the first original volume V₁ and the B cross-section image 1423 of the second original volume V₂. The B cross-section image 1450 of the merged volume 1430 may be generated by merging the B cross-section image 1413 of the first original volume V₁ and the A cross-section image 1421 of the second original volume V₂. The C cross-section image 1460 of the merged volume 1430 may be generated by merging the C cross-section image 1415 of the first original volume V₁ and the C cross-section image 1425 of the second original volume V₂.

Referring to FIG. 14B, the ultrasound imaging apparatus 40 may generate a cross-sectional image of a merged volume to be preferentially displayed over other cross-sectional images of the original volume, based on the reception of the user's input to select a cross-sectional image of an original volume.

For example, based on the reception of the user's input to select the A cross-section image 1421 of the second original volume V₂, the ultrasound imaging apparatus 40 may identify the B cross-section image 1450 of FIG. 14A of the merged volume as a cross-sectional image in which data of the A cross-section image 1421 of the second original volume V₂ is merged.

The ultrasound imaging apparatus 40 may generate a new B cross-section image 1455 of the merged volume 1430 by merging data of the B cross-section area of the first original volume V₁ and data of the A cross-section area of the second original volume V₂ and selecting data of the second original volume V₂ for an area where data overlap. The ultrasound imaging apparatus 40 may display the B cross-section image 1455 that is newly generated.

According to an embodiment, based on the reception of the user′s input to select the merged volume 1430, the ultrasound imaging apparatus 40 may display again the B cross-section image 1450 of FIG. 14A of the merged volume 1430.

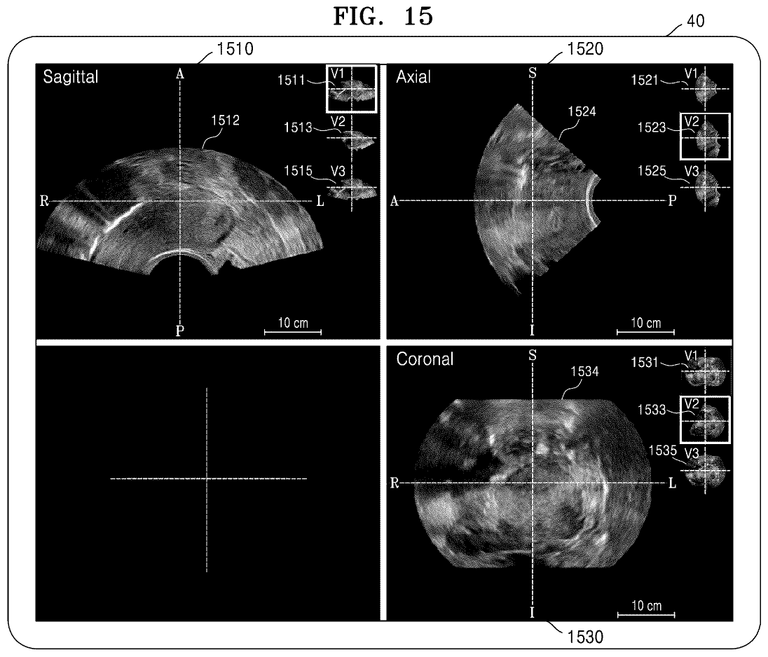

FIG. 15 is a diagram showing a method, performed by the ultrasound imaging apparatus 40, of displaying a cross-sectional image of an original volume corresponding to the cross-section of a merged volume, according to an embodiment.

Referring to FIG. 15, the ultrasound imaging apparatus 40 may provide a user interface for displaying a cross-sectional image of an original volume corresponding to the cross-section of a merged volume. The user interface for displaying a cross-sectional image of an original volume may include a thumbnail image of the cross-sectional image of the original volume.

The ultrasound imaging apparatus 40 may obtain a first original volume V₁, a second original volume V₂, and a third original volume V₃, through three three-dimensional scans of an object. Furthermore, the ultrasound imaging apparatus 40 may merge the other volumes based on the coordinate system of the reference volume.

The ultrasound imaging apparatus 40 may display a sagittal plane image (not shown) of the merged volume in a sagittal plane display area 1510. The sagittal plane of the merged volume may mean a cross-section indicating a sagittal plane of the object among the cross-sections of the merged volume.

The ultrasound imaging apparatus 40 may provide sagittal plane images of the first to third original volumes V1, V2, and V3 which constitute the sagittal plane image of the merged volume.

The ultrasound imaging apparatus 40 may display each of thumbnail images 1511, 1513, and 1515 of the original volume through a user interface for displaying sagittal plane images of original volumes.

The ultrasound imaging apparatus 40 may obtain the sagittal plane images of the original volumes transformed with respect to a reference coordinate system, based on the position of the sagittal plane of the merged volume in the reference coordinate system. Furthermore, the ultrasound imaging apparatus 40 may generate thumbnail images of the obtained sagittal plane images. For example, the ultrasound imaging apparatus 40 may obtain a sagittal plane image 1512 of the first original volume V₁ transformed with respect to the reference coordinate system, and generate the thumbnail image 1511 of the sagittal plane image 1512 of the first original volume V₁ that is obtained.

The ultrasound imaging apparatus 40 may display, in the sagittal plane display area 1510, the sagittal plane image 1512 of the first original volume V₁ that is selected based on the reception of the user's input to select the thumbnail image 1511 of the first original volume V₁ from among the thumbnail images 1511, 1513, and 1515 of the original volume indicating a sagittal plane.

The ultrasound imaging apparatus 40 may display only the sagittal plane image 1512 of the first original volume V₁ that is selected, in the sagittal plane display area 1510. Furthermore, although not illustrated in FIG. 15, the ultrasound imaging apparatus 40 may generate a new sagittal plane image of the merged volume so as to preferentially display data of the first original volume V₁ that is selected, and display the generated sagittal plane image of the merged volume, in the sagittal plane display area 1510. For example, when merging the sagittal plane image of the first original volume V₁, the sagittal plane image of the second original volume V₂, and the sagittal plane image of the third original volume V₃, by merging the sagittal plane images so as to preferentially display the image of first original volume V₁, a new sagittal plane image of the merged volume may be generated. In this case, the ultrasound imaging apparatus 40 may merge the sagittal plane images without blending. Furthermore, according to an embodiment, the ultrasound imaging apparatus 40 may blend only a boundary where data of the first original volume V₁ meets data of another original volume.

The ultrasound imaging apparatus 40 may display the original sagittal plane image of the merged volume in the sagittal plane display area 1510 based on the reception of the user's input to cancel the selection of the thumbnail image 1511 of the first original volume V₁.

Likewise, the ultrasound imaging apparatus 40 may display an axial image 1524 of the second original volume V₂ that is selected, in an axial plane display area 1520, based on the reception of the user's input to select a thumbnail image 1523 of the second original volume V₂ among thumbnail images 1521, 1523, and 1525 of the original volume indicating the axial plane of the object.

Furthermore, the ultrasound imaging apparatus 40 may display a coronal plane image 1534 of the second original volume V₂ that is selected, in a coronal plane display area 1530, based on the reception of the user's input to select a thumbnail image 1533 of the second original volume V2 among thumbnail images 1531, 1533, and 1535 of the original volume indicating the coronal plane of the object.

Accordingly, the ultrasound imaging apparatus 40 may provide, for a cross-section of interest of the object, not only a cross-sectional image of a merged volume, but also cross-sectional images of original volumes constituting the cross-sectional image of the merged volume.

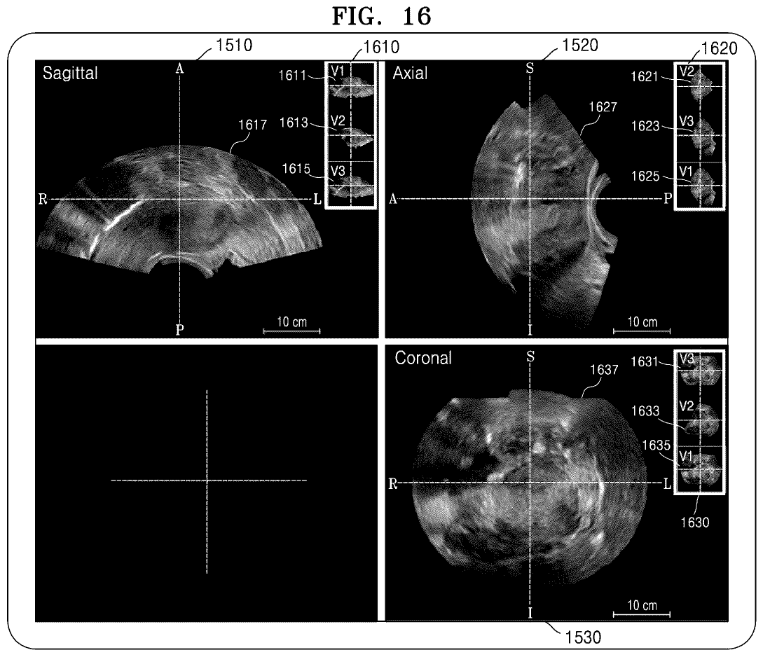

FIG. 16 is a diagram showing a method, performed by the ultrasound imaging apparatus 40, of providing a cross-sectional image of a merged volume based on priorities of original volumes, according to an embodiment.

Referring to FIG. 16, the ultrasound imaging apparatus 40 may receive a user's input to set priorities of original volumes. The ultrasound imaging apparatus 40 may update a cross-sectional image of a merged volume based on the set priorities of the original volumes.

The ultrasound imaging apparatus 40 may display user interface 1610, 1620, and 1630 for setting the priorities of the original volumes. For example, the ultrasound imaging apparatus 40 may display thumbnail images 1611, 1613, and 1615 of the original volumes indicating the cross-sections of the object. The ultrasound imaging apparatus 40 may receive a user's input to set the order of the thumbnail images 1611, 1613, and 1615 of the original volumes. The ultrasound imaging apparatus 40 may determine the priorities of the original volumes in the set order of the thumbnail images 1611, 1613, and 1615.

In the sagittal plane display area 1510 of FIG. 16, the ultrasound imaging apparatus 40 may receive a user′s input to set the order of thumbnail images 1611, 1613, and 1615 through a user interface 1610. The ultrasound imaging apparatus 40 may determine priorities in the order of the first original volume V₁, the second original volume V₂, and the third original volume V₃ according to the set order of the thumbnail images 1611, 1613, and 1615.

The ultrasound imaging apparatus 40 may newly generate a sagittal plane image 1617 so as to preferentially display data of the first original volume V₁, data of the second original volume V₂, and data of the third original volume V₃, for the area where data of the original volumes overlap. For example, the ultrasound imaging apparatus 40 may generate a new sagittal plane image 1617 of the merged volume by merging the sagittal plane images so as to preferentially display an image of the first original volume V₁, an image of the second original volume V₂, and an image of the third original volume V₃ in such an order. In this case, the ultrasound imaging apparatus 40 may merge the sagittal plane images without blending. Furthermore, according to an embodiment, the ultrasound imaging apparatus 40 may blend only a boundary where data of different original volumes meet.

The ultrasound imaging apparatus 40 may change the sagittal plane image (not shown) of the merged volume that has been merged without priority to the sagittal plane image 1617 that is newly generated.

Likewise, the ultrasound imaging apparatus 40 may receive a user′s input to set priorities in the order of the second original volume V₂, the third original volume V₃, and the first original volume V₁ by using thumbnail images 1621, 1623, and 1625 of the original volume indicating the axial plane of the object. The ultrasound imaging apparatus 40 may newly generate an axial image 1627 and display the axial image 1627 that is newly generated, so as to preferentially display data of the second original volume V₂, data of the third original volume V₃, and data of the first original volume V₁ in such an order, for the area where data of the original volumes overlap.

Furthermore, the ultrasound imaging apparatus 40 may receive a user's input to set priorities in the order of the third original volume V₃, the second original volume V₂, and the first original volume V₁, by using thumbnail images 1631, 1633, and 1635 of the original volume indicating the coronal plane of the object. The ultrasound imaging apparatus 40 may newly generate a coronal plane image 1637 so as to preferentially display data of the third original volume V₃, data of the second original volume V₂, and data of the first original volume V₁ in such an order, for the area where data of the original volumes overlap, and display the coronal plane image 1637 that is newly generated.

Accordingly, the ultrasound imaging apparatus may provide a cross-sectional image of a merged volume newly generated according to the set priorities of original volumes for a cross-section of interest of an object.

A machine-readable storage medium may be provided in the form of a non-transitory storage medium. The non-transitory may mean that a storage medium is a tangible device, not including a signal, for example, an electromagnetic wave. However, the term does not distinguish a case of semi-permanently storing data in a storage medium from a case of temporarily storing data. In an example, a non-transitory storage medium may include a buffer in which data is temporarily stored.

According to an embodiment, the method according to various embodiments may be provided by being included in a computer program product. A computer program product as goods may be dealt between a seller and a buyer. A computer program product may be distributed in the form of a device-readable storage medium (e.g., a compact disc read only memory (CD-ROM)), or through an application store or directly online between two user devices (e.g., smartphones) (e.g., download or upload). For online distribution, at least part of a computer program product (e.g., a downloadable application) may be at least temporarily stored or generated on a device-readable storage medium such as a manufacturer's server, a server of the application store, or a memory of a relay server.

What is claimed is:

1. An ultrasound imaging apparatus comprising:
an ultrasound transceiving module;
a memory storing instructions; and
at least one processor comprising processing circuitry,
wherein, when the instructions are individually or collectively executed by the at least one processor, the ultrasound imaging apparatus is configured to:
obtain a plurality of original volumes through the ultrasound transceiving module;
generate a merged volume by merging the plurality of original volumes;
display a merged volume cross-section image indicating a cross-sectional area of interest within the merged volume;
receive a user's input to select one of the plurality of original volumes; and
based on a position of the cross-sectional area of interest within the merged volume, display an original volume cross-section image of the selected original volume corresponding to the merged volume cross-section image.

2. The ultrasound imaging apparatus of claim 1, wherein, when the instructions are individually or collectively executed by the at least one processor, the ultrasound imaging apparatus is configured to:
based on the position of the cross-sectional area of interest within the merged volume, obtain the original volume cross-section image of the selected original volume corresponding to the merged volume cross-section image; and
display the obtained original volume cross-section image.

3. The ultrasound imaging apparatus of claim 1, wherein, when the instructions are individually or collectively executed by the at least one processor, the ultrasound imaging apparatus is configured to update the merged volume cross-section image so as to preferentially display data of the selected original volume for an area where the data of the selected original volume overlaps data of other original volumes in the cross-sectional area of interest within the merged volume.

4. The ultrasound imaging apparatus of claim 1, wherein, when the instructions are individually or collectively executed by the at least one processor, the ultrasound imaging apparatus is configured to:
receive a user's input to set priorities of the plurality of original volumes; and
update the merged volume cross-section image so as to preferentially display data of an original volume with relatively high priority for an area where data of the plurality of original volumes overlap.

5. The ultrasound imaging apparatus of claim 1, wherein, when the instructions are individually or collectively executed by the at least one processor, the ultrasound imaging apparatus is configured to receive a user's input to set a reference volume from among the plurality of original volumes; and merge the plurality of original volumes based on a coordinate system of the reference volume.

6. The ultrasound imaging apparatus of claim 1, wherein, when the instructions are individually or collectively executed by the at least one processor, the ultrasound imaging apparatus is configured to receive a user's input to set the cross-sectional area of interest within the merged volume.

7. The ultrasound imaging apparatus of claim 1, wherein the cross-sectional area of interest comprises a plurality of cross-sectional areas of interest, and
when the instructions are individually or collectively executed by the at least one processor, the ultrasound imaging apparatus is configured to:
display a plurality of merged volume cross-section images indicating the plurality of cross-sectional areas of interest within the merged volume; and
display a plurality of original volume cross-section images of the selected original volume corresponding to the plurality of merged volume cross-section images.

8. The ultrasound imaging apparatus of claim 7, wherein the plurality of merged volume cross-section images comprise a plane A, a plane B, and a plane C of the merged volume.

9. The ultrasound imaging apparatus of claim 7, wherein the plurality of original volumes comprise a plurality of volumes obtained by scanning a brain of a fetus in different directions, and
the plurality of merged volume cross-section images comprise images of a plurality of predetermined cross-sectional areas of interest within the merged volume.

10. The ultrasound imaging apparatus of claim 1, wherein, when the instructions are individually or collectively executed by the at least one processor, the ultrasound imaging apparatus is configured to display a user interface for selecting one of the plurality of original volumes, and
the user interface comprises cross-sectional images of the plurality of original volumes as identification information of the plurality of original volumes.

11. A method, performed by an ultrasound imaging apparatus, of visualizing an ultrasound merged volume, the method comprising:
obtaining a plurality of original volumes;
generating a merged volume by merging the plurality of original volumes;
displaying a merged volume cross-section image indicating a cross-sectional area of interest within the merged volume;
receiving a user's input to select one of the plurality of original volumes; and
displaying an original volume cross-section image of the selected original volume corresponding to the merged volume cross-section image based on a position of the cross-sectional area of interest within the merged volume.

12. The method of claim 11, wherein the displaying of the original volume cross-section image of the selected original volume corresponding to the merged volume cross-section image based on the position of the cross-sectional area of interest within the merged volume comprises:
obtaining an original volume cross-section image of the selected original volume corresponding to the merged volume cross-section image based on the position of the cross-sectional area of interest within the merged volume; and
displaying the obtained original volume cross-section image.

13. The method of claim 11, wherein the displaying of the original volume cross-section image of the selected original volume corresponding to the merged volume cross-section image based on the position of the cross-sectional area of interest within the merged volume comprises updating the merged volume cross-section image so as to preferentially display data of the selected original volume for an area where the data of the selected original volume overlaps data of other original volumes in the cross-sectional area of interest within the merged volume.

14. The method of claim 11, further comprising:
receiving a user's input to set priorities of the plurality of original volumes; and
updating the merged volume cross-section image so as to preferentially display data of an original volume with relatively high priority for an area where data of the plurality of original volumes overlap.

15. The method of claim 11, further comprising receiving a user's input to set a reference volume from among the plurality of original volumes; and merging the plurality of original volumes based on a coordinate system of the reference volume.

16. The method of claim 11, further comprising receiving a user's input to set the cross-sectional area of interest within the merged volume.

17. The method of claim 11, wherein the cross-sectional area of interest comprises a plurality of cross-sectional areas of interest,
the method further comprising:
displaying a plurality of merged volume cross-section images indicating the plurality of cross-sectional areas of interest within the merged volume; and
displaying a plurality of original volume cross-section images of the selected original volume corresponding to the plurality of merged volume cross-section images.

18. The method of claim 17, wherein the plurality of merged volume cross-section images comprise a plane A, a plane B, and a plane C of the merged volume.

19. The method of claim 17, wherein the plurality of original volumes comprise a plurality of volumes obtained by scanning a brain of a fetus in different directions, and
the plurality of merged volume cross-section images comprise images of a plurality of predetermined cross-sectional areas of interest within the merged volume.

20. The method of claim 11, further comprising displaying a user interface for selecting one of the plurality of original volumes, wherein the user interface comprises cross-sectional images of the plurality of original volumes as identification information of the plurality of original volumes.
